(19) United States
(12) Patent Application Publication
(10) Pub. No.: US 2009/0318549 A1
(43) Pub. Date: Dec. 24, 2009

(54) COMBINATION TREATMENT OF GLAUCOMA

(75) Inventor: Zuhal Butuner, Oakville (CA)

Correspondence Address: SCHWEGMAN, LUNDBERG & WOESSNER, P.A., P.O. BOX 2938, MINNEAPOLIS, MN, 55402, US

(73) Assignee: QLT Plug Delivery, Inc., Menlo Park, CA (US)

(21) Appl. No.: 12/490,923

(22) Filed: Jun. 24, 2009

Related U.S. Application Data
(60) Provisional application No. 61/075,284, filed on Jun. 24, 2008.

(51) Int. Cl. A61K 31/215; A61P 27/06

(52) U.S. Cl. 514/530

(57) ABSTRACT

The methods described herein provide reduction of intraocular pressure by administering a sustained release formulation including latanoprost and a pharmaceutically acceptable vehicle and administering an eye drop adjunctive composition to the eye of a patient. The sustained release formulation can release latanoprost continuously for at least 90 days from a punctum plug delivery system. The eye drop adjunctive composition can also include latanoprost.

### CLAIM OF PRIORITY

Benefit of priority is hereby claimed to U.S. Provisional Patent Application Ser. No. 61/075,284, filed on Jun. 24, 2008 and entitled Combination Treatment of Glaucoma, the specification of which is herein incorporated by reference in its entirety.

### BACKGROUND OF THE INVENTION

Glaucoma is a collection of disorders characterized by progressive visual field loss due to optic nerve damage. It is the leading cause of blindness in the United States, affecting 1-2% of individuals aged 60 and over. Although there are many risk factors associated with the development of glaucoma (age, race, myopia, family history, and injury), elevated intraocular pressure, also known as ocular hypertension, is the only risk factor successfully manipulated and correlated with the reduction of glaucomatous optic neuropathy. Public health figures estimate that 2.5 million Americans manifest ocular hypertension.

In order to manage glaucoma and ocular hypertension, topical drugs are often required to be administered to the eye. However, administration and compliance are often problematic. Therefore, improved drug delivery systems and administration protocols are needed.

### SUMMARY OF THE INVENTION

The present invention provides methods to reduce intraocular pressure in a patient. The methods include administering a sustained release formulation including latanoprost and a pharmaceutically acceptable vehicle and administering an eye drop adjunctive composition to the eye of the patient. In some embodiments, the sustained release formulation releases latanoprost continuously for at least 90 days from a punctum plug delivery system.

In some embodiments, the eye drop adjunctive composition includes an ocular hypotensive drug. Ocular hypotensive drugs include carbonic anhydrase inhibitors, beta blockers, alpha-adrenergic agents, prostaglandin analogues, miotics and epinephrine compounds. In one embodiment, the ocular hypotensive drug is latanoprost, a prostaglandin analogue. In one embodiment, the eye drop adjunctive composition contains 1.5 micrograms of latanoprost per drop.

The eye drop adjunctive composition can be administered once daily, twice daily, three times daily, or more. The eye drop adjunctive composition can be administered once every other day or once every three days. In some embodiments, the eye drop adjunctive composition is administered for less than about 30 days, less than about 20 days, less than about 10 days, or less than about 5 days.

The eye drop adjunctive composition may be administered starting on about the same day that the punctum plug delivery system is inserted into at least one punctum of the patient, about the day after the punctum plug delivery system is inserted, about two days after the punctum plug delivery system is inserted, about three days after the punctum plug delivery system is inserted, about four days after the punctum plug delivery system is inserted, about five days after the punctum plug delivery system is inserted, about six days after the punctum plug delivery system is inserted, about one week after the punctum plug delivery system is inserted, about two weeks after the punctum plug delivery system is inserted, about three weeks after the punctum plug delivery system is inserted, or about four weeks after the punctum plug delivery system is inserted. In some embodiments, the eye drop adjunctive composition is administered within about one week, within about two weeks, within about three weeks, within about four weeks, or within about five weeks after the punctum plug delivery system is inserted into at least one punctum of the patient.

In one embodiment, the eye drop adjunctive composition is administered once daily, starting about 90 days after the punctum plug delivery system is inserted into a punctum of the patient. The eye drop adjunctive composition may also be administered after removal of the punctum plug delivery system or before the punctum plug delivery system is inserted. In one embodiment, the eye drop adjunctive composition is administered starting approximately five days before the punctum plug delivery system is inserted into a punctum of the patient. In other embodiments, the eye drop adjunctive composition is administered after a first punctum plug delivery system is removed and before a second punctum plug delivery system is inserted into a punctum of the patient.

In some embodiments, the punctum plug delivery system releases between about 25 ng/day and about 250 ng/day of latanoprost. The intraocular pressure before administering the latanoprost and eye drop adjunctive composition may be about 22 mm Hg, about 21 mm Hg, about 20 mm Hg, about 19 mm Hg, about 18 mm Hg, or about 17 mm Hg, or lower. In some embodiments, the intraocular pressure before administering the latanoprost and eye drop adjunctive composition is about 23 mm Hg, about 24 mm Hg, about 25 mm Hg, about 26 mm Hg, or higher. In some embodiments, the intraocular pressure before administering the latanoprost and eye drop adjunctive composition is at least 19 mm Hg, at least 20 mm Hg, at least 21 mm Hg, at least 22 mm Hg, at least 23 mm Hg, at least 24 mm Hg, or at least 25 mm Hg. The intraocular pressure can be reduced to about 10 mm Hg, about 11 mm Hg, about 12 mm Hg, about 13 mm Hg, about 14 mm Hg, about 15 mm Hg, about 16 mm Hg, about 17 mm Hg, about 18 mm Hg, about 19 mm Hg, or about 20 mm Hg, after administering the latanoprost and eye drop adjunctive composition. In some embodiments, the intraocular pressure is reduced at least 2 mm Hg, at least 3 mm Hg, at least 4 mm Hg, at least 5 mm Hg, at least 6 mm Hg, at least 7 mm Hg, at least 8 mm Hg, at least 9 mm Hg, at least 10 mm Hg, at least 11 mm Hg, at least 12 mm Hg, at least 13 mm Hg, at least 14 mm Hg, or at least 15 mm Hg after administering the latanoprost and eye drop adjunctive composition.

In certain embodiments, the reduction in intraocular pressure is maintained for a continuous period of time. This continuous period of time may be up to about 7 days, up to about 14 days, up to about 21 days, up to about 28 days, up to about 52 days, up to about 88 days, or up to about 105 days. In one embodiment, the reduction in intraocular pressure is maintained for a continuous period of time of at least about 90 days.

In some embodiments, the reduction in intraocular pressure after administering the latanoprost and eye drop adjunctive composition is at least about 10%, at least about 12%, at least about 15%, at least about 17%, at least about 20%, at least about 25%, at least about 30%, or at least about 35%, or higher.

The intraocular pressure may be reduced within about 1 day, within about 2 days, within about 3 days, within about 4 days, within about 5 days, within about 6 days, within about 7 days, within about 8 days, within about 9 days, or within about 10 days after administering the latanoprost and eye drop adjunctive composition. In one embodiment, the intraocular pressure is reduced by at least 10% by about 1 day after latanoprost and eye drop adjunctive composition administration is initiated.

The invention also provides a punctum plug delivery system containing at least 3 micrograms latanoprost, at least 10 micrograms latanoprost, at least 20 micrograms latanoprost, at least 30 micrograms latanoprost, or at least 40 micrograms latanoprost. In some embodiments, the punctum plug delivery system contains about 3.5 micrograms latanoprost, about 14 micrograms latanoprost, or about 21 micrograms latanoprost. In some embodiments, the punctum plug delivery system includes a cavity configured to house the sustained release agent supply in the form of a drug core.

The pharmaceutically acceptable vehicle of the sustained release formulation can be a sustained release matrix. In some embodiments, the sustained release matrix is a non-biodegradable polymer. The non-biodegradable polymer may be silicone.

The punctum plug delivery system can be inserted into at least one punctum of the patient, into one punctum of each of both eyes of the patient, or into one punctum of one eye. The punctum plug delivery system can be inserted into an upper punctum, into a lower punctum, or into each of the upper and lower puncta. In some embodiments, the punctum plug delivery system can be inserted into at least 2, at least 3, or at least 4 puncta of the patient.

The intraocular pressure reduced by the methods of the instant invention can be associated with ocular hypertension. This ocular hypertension may be associated with glaucoma. Glaucoma includes primary open angle glaucoma, angle closure glaucoma, normal tension glaucoma and secondary glaucoma.

The invention described herein also provides methods to treat elevated intraocular pressure by inserting a punctum plug delivery system into at least one punctum of a patient and administering an eye drop adjunctive composition to an eye of the patient, wherein the punctum plug delivery system includes a sustained release agent supply containing about 14 micrograms of latanoprost, wherein the punctum plug delivery system remains inserted for at least about 90 days, and wherein the eye drop adjunctive composition is administered for up to about 14 days. In some embodiments, the eye drop adjunctive composition is administered for about ten days, about five days, or about one day.

Also contemplated by the invention are methods to treat elevated glaucoma-associated intraocular pressure by inserting a punctum plug delivery system into at least one punctum of a subject and administering an eye drop adjunctive composition to an eye of the subject. In one embodiment, the punctum plug delivery system has a plug body and a latanoprost insert and the eye drop adjunctive composition includes latanoprost. In one embodiment, the punctum plug delivery system provides the sustained release of latanoprost to the subject. The release of latanoprost from the punctum plug delivery system and the administration of the eye drop adjunctive latanoprost composition together result in a reduction in the intraocular pressure of the associated eye of at least 6 mm Hg. The punctum plug delivery system releases latanoprost during a continuous period of time of at least about 7 days, at least about 28 days, at least about 52 days, or at least about 88 days following insertion of the implant, and the eye drop adjunctive composition is administered for approximately five days following insertion of the implant.

The instant invention also provides methods to treat glaucoma in a subject in need thereof, by inserting a punctum plug delivery system into at least one punctum of the subject in a single insertion procedure and administering a latanoprost eye drop adjunctive composition to the corresponding eye of the subject at least once; wherein the punctum plug delivery system includes a plug body and a latanoprost insert; and wherein the punctum plug delivery system provides the sustained release of latanoprost to the subject for at least about 90 days.

Also contemplated by the invention is a kit having a first container including the described punctum plug delivery system, a second container including the described eye drop adjunctive composition, and instructions for use.

### BRIEF DESCRIPTION OF THE FIGURES

In the drawings, like numerals can be used to describe similar components throughout the several views. The drawings illustrate generally, by way of example, but not by way of limitation, various embodiments discussed in the present document.

FIG. 1 illustrates an example of a cross-sectional view of a punctum plug configured to be retained at least partially within a lacrimal punctum or canalicular anatomy.

FIG. 2A illustrates an example of an isometric view of a punctum plug configured to be retained at least partially within a lacrimal punctum or canalicular anatomy.

FIG. 2B illustrates an example of a cross-sectional view of a punctum plug taken along a line parallel to a longitudinal axis of the plug, such as along line 2B-2B of FIG. 2A.

FIG. 2C illustrates an example of a cross-sectional view of another punctum plug taken along a line parallel to a longitudinal axis of the plug.

FIG. 3A illustrates an example of an isometric view of a punctum plug configured to be retained at least partially within a lacrimal punctum or canalicular anatomy.

FIG. 3B illustrates an example of a cross-sectional view of a punctum plug taken along a line parallel to a longitudinal axis of the plug, such as along line 3B-3B of FIG. 3A, and a dilation of a plug-receiving anatomical tissue structure.

FIG. 4A illustrates an example of an isometric view of a punctum plug configured to be retained at least partially within a lacrimal punctum or canalicular anatomy.

FIG. 4B illustrates an example of a cross-sectional view of a punctum plug taken along a line parallel to a longitudinal axis of the plug, such as along line 4B-4B of FIG. 4A.

### DETAILED DESCRIPTION OF THE INVENTION

### Definitions:

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B.” “B but not A,” and “A and B,” unless otherwise indicated.

As used herein, the term “about” is used to refer to an amount that is approximately, nearly, almost, or in the vicinity of being equal to a stated amount.

As used herein, the phrase “consisting essentially of” limits a composition to the specified materials or steps and those additional, undefined components that do not materially affect the basic and novel characteristic(s) of the composition.

As used herein, the term “continuous” or “continuously” means unbroken or uninterrupted. For example, continuously administered active agents are administered over a period of time without interruption.

As used herein, the term “eye” refers to any and all anatomical tissues and structures associated with an eye. The eye is a spherical structure with a wall having three layers: the outer sclera, the middle choroid layer and the inner retina. The sclera includes a tough fibrous coating that protects the inner layers. It is mostly white except for the transparent area at the front, the cornea, which allows light to enter the eye. The choroid layer, situated inside the sclera, contains many blood vessels and is modified at the front of the eye as the pigmented iris. The biconvex lens is situated just behind the pupil. The chamber behind the lens is filled with vitreous humour, a gelatinous substance. The anterior and posterior chambers are situated between the cornea and iris, respectively and filled with aqueous humour. At the back of the eye is the light-detecting retina. The cornea is an optically transparent tissue that conveys images to the back of the eye. It includes avascular tissue to which nutrients and oxygen are supplied via bathing with lacrimal fluid and aqueous humour as well as from blood vessels that line the junction between the cornea and sclera. The cornea includes one pathway fro the permeation of drugs into the eye. Other anatomical tissue structures associated with the eye include the lacrimal drainage system, which includes a secretory system, a distributive system and an excretory system. The secretory system comprises secretors that are stimulated by blinking and temperature change due to tear evaporation and reflex secretors that have an efferent parasympathetic nerve supply and secrete tears in response to physical or emotional stimulation. The distributive system includes the eyelids and the tear meniscus around the lid edges of an open eye, which spread tears over the ocular surface by blinking, thus reducing dry areas from developing.

As used herein, the term “implant” refers to a structure that can be configured to contain or be impregnated with a drug core or a drug matrix, such as those as disclosed in this patent document and in WO 07/115,261, which is herein incorporated by reference in its entirety, which is capable of releasing a quantity of active agent, such as latanoprost, into tear fluid for a sustained release period of time when the structure is implanted at a target location along the path of the tear fluid in the patient. The terms “implant,” “plug” and “punctum plug” are meant herein to refer to similar structures. Likewise, the terms “implant body” and “plug body” are meant herein to refer to similar structures. The terms “ocular implant” and “punctum plug delivery system” refer to similar structures and are used interchangeably herein. The implants described herein may be inserted into the punctum of a subject, or through the punctum into the canaliculus. The implant may be also the drug core or drug matrix itself, which is configured for insertion into the punctum without being housed in a carrier such as a punctal plug occluder, for example having a polymeric component and a latanoprost component with no additional structure surrounding the polymeric component and latanoprost component.

As used herein, a “pharmaceutically acceptable vehicle” is any physiological vehicle known to those of ordinary skill in the art useful in formulating pharmaceutical compositions. Suitable vehicles include polymeric matrices, sterile distilled or purified water, isotonic solutions such as isotonic sodium chloride or boric acid solutions, phosphate buffered saline (PBS), propylene glycol and butylene glycol. Other suitable vehicular constituents include phenylmercuric nitrate, sodium sulfate, sodium sulfite, sodium phosphate and monosodium phosphate. Additional examples of other suitable vehicle ingredients include alcohols, fats and oils, polymers, surfactants, fatty acids, silicone oils, humectants, moisturizers, viscosity modifiers, emulsifiers and stabilizers. The compositions may also contain auxiliary substances, i.e. antimicrobial agents such as chlorobutanol, parabans or organic mercurial compounds; pH adjusting agents such as sodium hydroxide, hydrochloric acid or sulfuric acid; and viscosity increasing agents such as methylcellulose. The final composition should be sterile, essentially free of foreign particles, and have a pH that allows for optimum drug stability.

As used herein, the term “punctum” refers to the orifice at the terminus of the lacrimal canaliculus, seen on the margins of the eyelids at the lateral extremity of the lacus lacrimalis. Puncta (plural of punctum) function to reabsorb tears produced by the lacrimal glands. The excretory part of the lacrimal drainage system includes, in flow order of drainage, the lacrimal puncta, the lacrimal canaliculi, the lacrimal sac and the lacrimal duct. From the lacrimal duct, tears and other flowable materials drain into a passage of the nasal system. The lacrimal canaliculi include an upper (superior) lacrimal canaliculus and a lower (inferior) lacrimal canaliculus, which respectively terminate in an upper and lower lacrimal punctum. The upper and lower punctum are slightly elevated at the medial end of a lid margin at the junction of the ciliary and lacrimal portions near a conjunctival sac. The upper and lower punctum are generally round or slightly ovoid openings surrounded by a connective ring of tissue. Each of the puncta leads into a vertical portion of their respective canaliculus before turning more horizontal at a canaliculus curvature to join one another at the entrance of the lacrimal sac. The canaliculi are generally tubular in shape and lined by stratified squamous epithelium surrounded by elastic tissue, which permits them to be dilated.

The terms “subject” and “patient” refer to animals such as mammals, including, but not limited to, primates (e.g., humans), cows, sheep, goats, horses, dogs, cats, rabbits, rats, mice and the like. In many embodiments, the subject or patient is a human.

A “therapeutic agent” can comprise a drug and may be any of the following or their equivalents, derivatives or analogs, including anti-glaucoma medications (e.g. ocular hypotensive drugs) including carbonic anhydrase inhibitors (CAIs, including but not limited to dorzolamide, brinzolamide, diamox, methazolamide, dorzolamide+timolol, acetazolamide, and dichlorphenamide); Beta blockers including but not limited to levobunolol (Betagan), timolol (Betimol, Timoptic), carteolol (Ocupress), betaxolol (Betoptic), atenolol (Tenormin), and metipranolol (OptiPranolol); Alpha-adrenergic agents including but not limited to apraclonidine (Iopidine) and brimonidine (Alphagan); Prostaglandin analogues including but not limited to: latanoprost (Xalatan), bimatoprost (Lumigan) and travoprost (Travatan); Miotics including but not limited to pilocarpine (Isopto Carpine, Pilocar); Epinephrine compounds; parasympathomimetics, hypotensive lipids, and combinations thereof; antimicrobial agents (e.g., antibiotic, antiviral, antiparacytic, antifungal, etc.); analgesics such as keterolac; corticosteroids or other anti-inflammatories (e.g., an NSAID such as diclofenac or naproxen); decongestants (e.g., vasoconstrictors); agents that prevent or modify an allergic response (e.g., antihistamines such as olopatadine, cytokine inhibitor, leucotriene inhibitor, IgE inhibitor, immunomodulator or immunosuppressants such as cyclosporin); mast cell stabilizers; cycloplegics or the like. Examples of conditions that may be treated with the therapeutic agent(s) include but are not limited to glaucoma, pre and post surgical treatments, ocular hypertension, dry eye and allergies. In some embodiments, the therapeutic agent may be a lubricant or a surfactant, for example a lubricant to treat dry eye.

Exemplary therapeutic agents include, but are not limited to thrombin inhibitors; antithrombogenic agents; thrombolytic agents; fibrinolytic agents; vasospasm inhibitors; vasodilators; antihypertensive agents; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflaTnmatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non steroidal anti-inflammatories (NSAIDs, such as salicylate, indomethacin, ibuprofen, diclofenac, flurbiprofen, piroxicam indomethacin, ibuprofen, naxopren, piroxicam and nabumetone). Such anti inflammatory steroids contemplated for use in the methodology of the present invention, include triamcinolone acetonide (generic name) and corticosteroids that include, for example, triamcinolone, dexamethasone, fluocinolone, cortisone, prednisolone, flumetholone, and derivatives thereof.); antiallergenics (such as sodium chromoglycate, antazoline, methapyriline, chlorpheniramine, cetrizine, pyrilamine, prophenpyridamine); anti proliferative agents (such as 1,3-cis retinoic acid, 5-fluorouracil, taxol, rapamycin, mitomycin C and cisplatin); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil3; immunological drugs (such as vaccines and immune stimulants); hormonal agents (such as estrogens, -estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotrapin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix; components; ACE inhibitors; free radical scavengers; chelators; antioxidants; anti polymerases; photodynamic therapy agents; gene therapy agents; and other therapeutic agents such as prostaglandins, antiprostaglandins, prostaglandin precursors, neuroprotectants such as lubezole, nimodipine and related compounds; and parasympathomimetrics such as pilocarpine, carbachol, physostigmine and the like.

The term “topical” refers to any surface of a body tissue or organ. A topical formulation is one that is applied to a body surface, such as an eye, to treat that surface or organ. Topical formulations include liquid drops such as eye drops; creams, lotions, sprays, emulsions, and gels. Topical formulations as used herein also include formulations that release therapeutic agents into the tears to result in topical administration to the eye.

As used herein, the term “treating” or “treatment” of a disease includes: (1) preventing the disease, i.e., causing the clinical symptoms of the disease not to develop in a subject that may be exposed to or predisposed to the disease but who does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

### Elevated Intraocular Pressure:

Ocular hypertension (OH) and primary open angle glaucoma (POAG) are caused by a build-up of aqueous humor in the anterior chamber primarily due to the eye's inability to properly drain aqueous fluid. The ciliary body, situated at the root of the iris, continuously produces aqueous humor. It flows into the anterior chamber and then drains via the angle between the cornea and iris through the trabecular meshwork and into a channel in the sclera. In the normal eye, the amount of aqueous humor being produced is equal to the amount that is draining out. However, in an eye in which this mechanism is compromised, intraocular pressure (IOP) rises. Elevated IOP represents a major risk factor for glaucomatous field loss. Results from several studies indicate that early intervention targeted at lowering intraocular pressure retards the progression of optic nerve damage and loss of visual fields that lead to decreased vision and blindness.

### Latanoprost:

A therapeutic agent for use in the methods described herein is latanoprost. Latanoprost is a prostaglandin F_{2α} analogue. Its chemical name is isopropyl-(Z)-7[(1R,2R,3R,5S)3,5-dihydroxy-2-[(3R)-3-hydroxy-5-phenylpentyl]cyclopentyl]-5-heptenoate. Its molecular formula is C₂₆H₄₀O₅ and its chemical structure is:

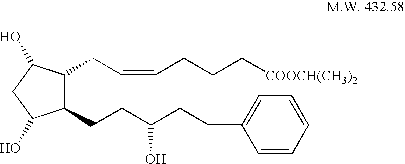

Latanoprost is a colorless to slightly yellow oil that is very soluble in acetonitrile and freely soluble in acetone, ethanol, ethyl acetate, isopropanol, methanol and octanol. It is practically insoluble in water.

Latanoprost is believed to reduce intraocular pressure (IOP) by increasing the outflow of aqueous humor. Studies in animals and man suggest that the main mechanism of action is increased uveoscleral outflow of aqueous fluid from the eyes. Latanoprost is absorbed through the cornea where the isopropyl ester prodrug is hydrolyzed to the acid form to become biologically active. Studies in man indicate that the peak concentration in the aqueous humor is reached about two hours after topical administration.

Xalatan® latanoprost ophthalmic solution is a commercially available product indicated for the reduction of elevated IOP in patients with open-angle glaucoma or ocular hypertension. The amount of latanoprost in the commercially available product Xalatan® is 50 micrograms per mL, approximately 1.5 micrograms/drop. Xalatan® is supplied as a 2.5 mL solution in a 5 mL clear, low density polyethylene (PET) bottle with a clear low density PET dropper tip, a turquoise high density PET screw cap, and a tamper-evident clear low density PET overcap. Inactive ingredients of Xalatan® are benzalkonium chloride (preservative), sodium chloride, sodium dihydrogen phosphate monohydrate, disodium hydrogen phosphate anhydrous, and water. As described above, eye drops, though effective, can be inefficient and require multiple applications to maintain the therapeutic benefit. Low patient compliance compounds these effects.

### Methods of Treatment:

The invention described herein provides methods to treat glaucoma, elevated intraocular pressure, and glaucoma-associated elevated intraocular pressure with a therapeutic agent or agents. In many embodiments, a method of treating an eye with latanoprost is provided. In some embodiments, the therapeutic agent is released to the eye over a sustained period of time. In an embodiment, the sustained period of time is approximately 90 days. In some embodiments, an eye drop adjunctive composition is additionally administered to the eye. In one embodiment, the eye drop adjunctive composition includes latanoprost. In some embodiments, the method comprises inserting through a punctum an implant having a body and a drug core so that the drug core is retained near the punctum. In some embodiments, the method comprises inserting through a punctum an implant having a body impregnated with a therapeutic agent and administering an eye drop adjunctive composition. An exposed surface of the drug core or impregnated body located near the proximal end of the implant contacts the tear or tear film fluid and the latanoprost migrates from the exposed surface to the eye over a sustained period of time while the drug core and body is at least partially retained within the punctum. In many embodiments, a method of treating an eye with latanoprost is provided, the method comprising inserting through a punctum into a canalicular lumen an implant having an optional retention structure so that the implant body is anchored to a wall of the lumen by the retention structure and administering an eye drop adjunctive composition. The implant releases effective amounts of latanoprost from a drug core or other agent supply into a tear or tear film fluid of the eye. In some embodiments, the drug core may be removed from the retention structure while the retention structure remains anchored within the lumen. A replacement drug core can then be attached to the retention structure while the retention structure remains anchored. At least one exposed surface of the replacement drug core releases latanoprost at therapeutic levels over a sustained period.

A replacement drug core can be attached to the retention structure approximately every 90 days to result in continuous release of the drug to the eye for a period of time of approximately 180 days, approximately 270 days, approximately 360 days, approximately 450 days, approximately 540 days, approximately 630 days, approximately 720 days, approximately 810 days or approximately 900 days. In some embodiments, a replacement plug can be inserted into the punctum approximately every 90 days to achieve release of the drug to the eye for extended periods of time, including up to about 180 days, about 270 days, about 360 days, about 450 days, about 540 days, about 630 days, about 720 days, about 810 days or about 900 days.

In other embodiments, a method for treating an eye with latanoprost is provided, the method comprising inserting a drug core or other implant body at least partially into at least one punctum of the eye and administering an eye drop adjunctive composition. The drug core may or may not be associated with a separate implant body structure. The drug core or agent-impregnated implant body provides sustained release delivery of latanoprost at therapeutic levels. In some embodiments, the sustained release delivery of latanoprost continues for up to 90 days.

In some embodiments, the eye drop adjunctive compositions are used on a limited time basis only. While not being bound by theory, it is believed that adjunctive eye drop therapy will serve to saturate certain receptors rapidly and optionally to maintain delivery especially during a period when sustained release from the punctum plug is in flux. In some embodiments, the receptors are prostaglandin receptors. In one embodiment, the receptors are prostaglandin F (FP) receptors. Subsequently, sustained and continuous delivery of a therapeutic agent via a punctum plug delivery system maintains saturation of the receptors and therapeutic effect.

The eye drop adjunctive composition can be administered once daily, twice daily, three times daily, or more. The eye drop adjunctive composition can be administered once every other day or once every three days. In some embodiments, the eye drop adjunctive composition is administered for less than about 30 days, less than about 20 days, less than about 10 days, or less than about 5 days. The eye drop adjunctive composition may be administered for a period of about one day, about two days, about three days, about four days, about five days, about six days, about seven days, about eight days, about nine days, about ten days, about eleven days, about twelve days, about thirteen days, about fourteen days, about fifteen days, about sixteen days, about seventeen days, about eighteen days, about nineteen days, or about twenty days.

The eye drop adjunctive composition may be administered starting on about the same day that the punctum plug delivery system is inserted into at least one punctum of the patient, about the day after the punctum plug delivery system is inserted, about two days after the punctum plug delivery system is inserted, about three days after the punctum plug delivery system is inserted, about four days after the punctum plug delivery system is inserted, about five days after the punctum plug delivery system is inserted, about six days after the punctum plug delivery system is inserted, about seven days after the punctum plug delivery system is inserted, about eight days after the punctum plug delivery system is inserted, about nine days after the punctum plug delivery system is inserted, about ten days after the punctum plug delivery system is inserted, about eleven days after the punctum plug delivery system is inserted, about twelve days after the punctum plug delivery system is inserted, about thirteen days after the punctum plug delivery system is inserted, about fourteen days after the punctum plug delivery system is inserted, about fifteen days after the punctum plug delivery system is inserted, about sixteen days after the punctum plug delivery system is inserted, about seventeen days after the punctum plug delivery system is inserted, about eighteen days after the punctum plug delivery system is inserted, about nineteen days after the punctum plug delivery system is inserted, about twenty days after the punctum plug delivery system is inserted, about twenty-one days after the punctum plug delivery system is inserted, about twenty-two days after the punctum plug delivery system is inserted, about twenty-three days after the punctum plug delivery system is inserted, about twenty-four days after the punctum plug delivery system is inserted, about twenty-five days after the punctum plug delivery system is inserted, about twenty-six days after the punctum plug delivery system is inserted, about twenty-seven days after the punctum plug delivery system is inserted, or about twenty-eight days after the punctum plug delivery system is inserted. The eye drop adjunctive composition may be administered starting about one week after the punctum plug delivery system is inserted, about two weeks after the punctum plug delivery system is inserted, about three weeks after the punctum plug delivery system is inserted, or about four weeks after the punctum plug delivery system is inserted. In some embodiments, the eye drop adjunctive composition is administered within about one week, within about two weeks, within about three weeks, within about four weeks, or within about five weeks after the punctum plug delivery system is inserted into at least one punctum of the patient. In one embodiment, the eye drop adjunctive composition is administered once daily, starting about 90 days after the punctum plug delivery system is inserted into a punctum of the patient. The eye drop adjunctive composition may also be administered after removal of the punctum plug delivery system or before the punctum plug delivery system is inserted. In one embodiment, the eye drop adjunctive composition is administered starting approximately five days before the punctum plug delivery system is inserted into a punctum of the patient. In other embodiments, the eye drop adjunctive composition is administered starting approximately one week or approximately two weeks or approximately one month or more before the punctum plug delivery system is inserted into a punctum of a patient. In other embodiments, the eye drop adjunctive composition is administered after a first punctum plug delivery system is removed and before a second punctum plug delivery system is inserted into a punctum of the patient.

In many embodiments, a method for treating an eye with latanoprost is provided, the method comprising inserting a distal end of an implant into at least one punctum of the eye and administering a latanoprost eye drop adjunctive composition. In some embodiment, a retention structure of the implant can be expanded so as to inhibit expulsion of the implant. The expansion of the retention structure can help to occlude a flow of tear fluid through the punctum. In some embodiments, the implant is configured such that, when implanted, an at least 45 degree angled intersection exists between a first axis, defined by a proximal end of the implant, and a second axis, defined by the distal end of the implant, to inhibit expulsion of the implant. Latanoprost is delivered from a proximal end of the implant to the tear fluid adjacent the eye. Delivery of the latanoprost is inhibited distally of the proximal end.

The methods of the invention provide sustained release of latanoprost in combination with eye drop adjunctive composition administration. In some embodiments, the latanoprost is released from the implant for at least one week, at least two weeks, at least three weeks, at least four weeks, at least five weeks, at least six weeks, at least seven weeks, at least eight weeks, at least nine weeks, at least ten weeks, at least eleven weeks, at least twelve weeks, at least thirteen weeks, at least fourteen weeks, at least fifteen weeks, or at least sixteen weeks. In an embodiment, the latanoprost is released for at least twelve weeks.

The amount of latanoprost associated with the implant may vary depending on the desired therapeutic benefit and the time during which the device is intended to deliver the therapy. Since the devices of the present invention present a variety of shapes, sizes and delivery mechanisms, the amount of drug associated with the device will depend on the particular disease or condition to be treated, and the dosage and duration that is desired to achieve the therapeutic effect. Generally, the amount of latanoprost is at least the amount of drug that, upon release from the device, is effective to achieve the desired physiological or pharmacological local or systemic effects.

Methods of inserting and removing the implant are known to those of skill in the art. For instance, tools for insertion and removal/extraction of implants are described in U.S. Patent Application No. 60/970,840 (filed Sep. 7, 2007 and entitled Insertion and Extraction Tools for Punctal Implants), the disclosure of which is incorporated herein in its entirety. Generally, for placement, the size of punctal plug to be used may be determined by using suitable magnification or, if provided, using a sizing tool that accompanies the punctal plug. The patient's punctum may be dilated if necessary to fit the punctal plug. A drop of proparacaine anesthetic may be used, preferably five minutes or more before insertion of the plug. A drop of lubricant may be applied if necessary to facilitate placement of the plug into the punctum. Using an appropriate placement instrument, the plug may be inserted into the superior or inferior punctum of the eye. After placement, the cap of the plug may be visible. This process may be repeated for the patient's other eye. For removal of the implant, small sterile surgical forceps may be used to securely grasp the plug at the tube section below the cap. Using a gentle tugging motion the plug may be gently retrieved.

### Implant:

In some embodiments, latanoprost is administered for a sustained period of time by a drug core which may or may not be associated with a separate implant body structure. In certain embodiments, an implant for use in the methods described herein is provided. The implant can be configured, when implanted at a target location along the path of tear fluid in the patient, to release a quantity of latanoprost into the tear fluid each day for a sustained release period of days, weeks, or months. The implant can be one of any number of different designs that releases latanoprost or other therapeutic agent for a sustained period of time. The disclosures of the following patent documents, which describe example implant embodiments for use in the methods of the current invention and methods of making those implants, are incorporated herein by reference in their entirety: U.S. Application Ser. No. 60/871,864 (filed Dec. 26, 2006 and entitled Nasolacrimal Drainage System Implants for Drug Therapy); U.S. application Ser. No. 11/695,537 (filed Apr. 2, 2007 and entitled Drug Delivery Methods, Structures, and Compositions for Nasolacrimal System); U.S. Application Ser. No. 60/787,775 (filed Mar. 31, 2006 and entitled Nasolacrimal drainage system implants for drug therapy); U.S. application Ser. No. 11/695,545 (filed Apr. 2, 2007 and entitled Nasolacrimal drainage system implants for drug therapy); U.S. Application Ser. No. 60/970,696 (filed Sep. 7, 2007 and entitled Expandable Nasolacrimal Drainage System Implants); U.S. Application Ser. No. 60/974,367 (filed Sep. 21, 2007 and entitled Expandable Nasolacrimal Drainage System Implants); U.S. Application Ser. No. 60/970,699 (filed Sep. 7, 2007 and entitled Manufacture of Drug Cores for Sustained Release of Therapeutic Agents); U.S. Application Ser. No. 60/970,709 (filed Sep. 7, 2007 and entitled Nasolacrimal Drainage System Implants for Drug Delivery); U.S. Application Ser. No. 60/970,720 (filed Sep. 7, 2007 and entitled Manufacture of Expandable Nasolacrimal Drainage System Implants); U.S. Application Ser. No. 60/970,755 (filed Sep. 7, 2007 and entitled Prostaglandin Analogues for Implant Devices and Methods); U.S. Application Ser. No. 60/970,820 (filed Sep. 7, 2007 and entitled Multiple Drug Delivery Systems and Combinations of Drugs with Punctal Implants); U.S. Application Ser. No. 61/049,347 (filed Apr. 30, 2008 and entitled Lacrimal Implants and Related Methods); U.S. Application Ser. No. 61/049,360 (filed Apr. 30, 2008 and entitled Lacrimal Implants and Related Methods); U.S. Application Ser. No. 61/036,816 (filed Mar. 14, 2008 and entitled Lacrimal Implants and Related Methods); U.S. Application Ser. No. 61/049,337 (filed Apr. 30, 2008 and entitled Lacrimal Implants and Related Methods); U.S. Application Ser. No. 61/049,329 (filed Apr. 30, 2008 and entitled Composite Lacrimal Insert); U.S. Application Ser. No. 61/049,317 (filed Apr. 30, 2008 and entitled Drug-Releasing Polyurethane Lacrimal Insert); U.S. Application Ser. No. 10/825,047 (filed Apr. 15, 2004 and entitled Drug Delivery via Punctal Plug); International Published Application WO 2006/014434; and International Application Serial No. PCT/US2007/065789 (filed Mar. 31, 2006, published as WO 2007/115259 and entitled Nasolacrimal Drainage System Implants for Drug Therapy).

Generally, the implant comprises a body. In some embodiments, the implant body has a distal end portion and a proximal end portion. The distal end portion of the body is at least partially insertable into the punctum to the canalicular lumen of the patient. The implant body may be at least impregnated with latanoprost or otherwise comprise latanoprost, such as within a matrix drug core that is inserted into the implant body. Exposure of the matrix drug core or impregnated body to the tear fluid causes an effective latanoprost release into the tear fluid over a sustained period. The implant may include a sheath disposed over at least a portion of the drug core to inhibit release of latanoprost from certain portions thereof. The implant body may have an outer surface configured to engage luminal wall tissues so as to inhibit expulsion when disposed therein. In many embodiments, an integral feedback or other projection is connected around the sheath near the proximal end of the drug core. In an embodiment, the feedback or other projection includes one or more wings sized to remain outside the punctum so as to retain the proximal end of the drug core near the punctum. In other embodiments, the feedback or other projection includes a full or partial (e.g., trimmed) collar connected around the sheath near the proximal end of the drug core. The collarcan be sized to remain outside the punctum so as to retain the proximal end of the drug core near the punctum.

In some embodiments, the implant comprises a drug core alone, lacking an additional structure surrounding the core. In some embodiments, the drug core comprises a latanoprost matrix comprising a pharmaceutically acceptable vehicle, for example, a non-bioabsorbable polymer, for example silicone in a non-homogenous mixture with the latanoprost. The non-homogeneous mixture in the drug core may comprise a silicone matrix saturated with the latanoprost or with inclusions of latanoprost. The inclusions in the drug core are a concentrated form of latanoprost, and the silicone matrix encapsulates the inclusions in the drug core. In specific embodiments, the latanoprost inclusions encapsulated within the silicone matrix comprise an inhomogeneous mixture of the inclusions encapsulated within the silicone matrix. The drug core inclusions can comprise latanoprost oil.

It is also within the scope of this invention to modify or adapt the implant device to deliver a high release rate, a low release rate, a bolus release, a burst release, or combinations thereof. A bolus of the drug may be released by the formation of an erodable polymer cap that is immediately dissolved in the tear or tear film. As the polymer cap comes in contact with the tear or tear film, the solubility properties of the polymer enable the cap to erode and the latanoprost is released all at once. A burst release of latanoprost can be performed using a polymer that also erodes in the tear or tear film based on the polymer solubility. In this example, the drug and polymer may be stratified along the length of the device so that as the outer polymer layer dissolves, the drug is immediately released. A high or low release rate of the drug could be accomplished by changing the solubility of the erodable polymer layer so that the drug layer released quickly or slowly. Other methods to release the latanoprost could be achieved through porous membranes, soluble gels (such as those in typical ophthalmic solutions), microparticle encapsulations of the drug, or nanoparticle encapsulation.

### Sheath Body:

The sheath body can comprise appropriate shapes and materials to control the migration of latanoprost from the drug core. In some embodiments, the sheath body houses the drug core and can fit snugly against the core. The sheath body is made from a material that is substantially impermeable to the latanoprost so that the rate of migration of latanoprost may be largely controlled by the exposed surface area of the drug core that is not covered by the sheath body. In many embodiments, migration of the latanoprost through the sheath body can be about one tenth of the migration of latanoprost through the exposed surface of the drug core, or less, often being one hundredth or less. In other words, the migration of the latanoprost through the sheath body is at least about an order of magnitude less that the migration of latanoprost through the exposed surface of the drug core. Suitable sheath body materials include polyimide, polyethylene terephthalate (hereinafter “PET”). The sheath body has a thickness, as defined from the sheath surface adjacent the core to the opposing sheath surface away from the core, from about 0.00025″ to about 0.0015″. The total diameter of the sheath that extends across the core ranges from about 0.2 mm to about 1.2 mm. The core may be formed by dip coating the core in the sheath material. Alternatively or in combination, the sheath body can comprise a tube and the core introduced into the sheath, for example as a liquid or solid that can be slid, injected or extruded into the sheath body tube. The sheath body can also be dip coated around the core, for example dip coated around a pre-formed core.

The sheath body can be provided with additional features to facilitate clinical use of the implant. For example, the sheath may receive a drug core that is exchangeable while the implant body, retention structure and sheath body remain implanted in the patient. The sheath body is often rigidly attached to the retention structure as described above, and the core is exchangeable while the retention structure retains the sheath body. In specific embodiments, the sheath body can be provided with external protrusions that apply force to the sheath body when squeezed and eject the core from the sheath body. Another drug core can then be positioned in the sheath body. In many embodiments, the sheath body or retention structure may have a distinguishing feature, for example a distinguishing color, to show placement such that the placement of the sheath body or retention structure in the canaliculus or other body tissue structure can be readily detected by the patient. The retention element or sheath body may comprise at least one mark to indicate the depth of placement in the canaliculus such that the retention element or sheath body can be positioned to a desired depth in the canaliculus based on the at least one mark.

### Retention Structure:

In many embodiments, a retention structure is employed to retain the implant in the punctum or canaliculus. The retention structure is attached to or integral with the implant body. The retention structure comprises an appropriate material that is sized and shaped so that the implant can be easily positioned in the desired tissue location, for example, the punctum or canaliculus. In some embodiments, the drug core may be attached to the retention structure via, at least in part, the sheath. In some embodiments, the retention structure comprises a hydrogel configured to expand when the retention structure is placed in the punctum. The retention structure can comprise an attachment member having an axially oriented surface. In some embodiments, expansion of the hydrogel can urge against the axially oriented surface to retain the hydrogel while the hydrogel is hydrated. In some embodiments, the attachment member can comprise at least one of a protrusion, a flange, a rim, or an opening through a portion of the retention structure. In some embodiments, the retention structure includes an implant body portion size and shape to substantially match an anatomy of the punctum and canaliculus.

The retention structure may have a size suitable to fit at least partially within the canalicular lumen. The retention structure can be expandable between a small profile configuration suitable for insertion and a large profile configuration to anchor the retention structure in the lumen, and the retention structure can be attached near the distal end of the drug core. In specific embodiments, the retention structure can slide along the drug core near the proximal end when the retention structure expands from the small profile configuration to the large profile configuration. A length of the retention structure along the drug core can be shorter in the large profile configuration than the small profile configuration.

In some embodiments, the retention structure is resiliently expandable. The small profile may have a cross section of no more than about 0.2 mm, and the large profile may have a cross section of no more than about 2.0 mm. The retention structure may comprise a tubular body having arms separated by slots. The retention structure can be disposed at least partially over the drug core.

In some embodiments, the retention structure is mechanically deployable and typically expands to a desired cross sectional shape, for example with the retention structure comprising a super elastic shape memory alloy such as Nitinol™. Other materials in addition to Nitinol™ can be used, for example resilient metals or polymers, plastically deformable metals or polymers, shape memory polymers, and the like, to provide the desired expansion. In some embodiments polymers and coated fibers available from Biogeneral, Inc. of San Diego, Calif. may be used. Many metals such as stainless steels and non-shape memory alloys can be used and provide the desired expansion. This expansion capability permits the implant to fit in hollow tissue structures of varying sizes, for example canaliculae ranging from 0.3 mm to 1.2 mm (i.e. one size fits all). Although a single retention structure can be made to fit canaliculae from 0.3 to 1.2 mm across, a plurality of alternatively selectable retention structures can be used to fit this range if desired, for example a first retention structure for canaliculae from 0.3 to about 0.9 mm and a second retention structure for canaliculae from about 0.9 to 1.2 mm. The retention structure has a length appropriate to the anatomical structure to which the retention structure attaches, for example a length of about 3 mm for a retention structure positioned near the punctum of the canaliculus. For different anatomical structures, the length can be appropriate to provide adequate retention force, e.g. 1 mm to 15 mm lengths as appropriate.

Although the implant body may be attached to one end of the retention structure as described above, in many embodiments the other end of the retention structure is not attached to the implant body so that the retention structure can slide over the implant body including the sheath body and drug core while the retention structure expands. This sliding capability on one end is desirable as the retention structure may shrink in length as the retention structure expands in width to assume the desired cross sectional width. However, it should be noted that many embodiments may employ a sheath body that does not slide in relative to the core.

In many embodiments, the retention structure can be retrieved from tissue. A projection, for example a hook, a loop, or a ring, can extend from a portion of the implant body to facilitate removal of the retention structure.

In some embodiments the sheath and retention structure can comprise two parts.

### Occlusive Element:

An occlusive element can be mounted to and expandable with the retention structure to inhibit tear flow. An occlusive element may inhibit tear flow through the lumen, and the occlusive element may cover at least a portion of the retention structure to protect the lumen from the retention structure. The occlusive element comprises an appropriate material that is sized and shaped so that the implant can at least partially inhibit, even block, the flow of fluid through the hollow tissue structure, for example lacrimal fluid through the canaliculus. The occlusive material may be a thin walled membrane of a biocompatible material, for example silicone, that can expand and contract with the retention structure. The occlusive element is formed as a separate thin tube of material that is slid over the end of the retention structure and anchored to one end of the retention structure as described above. Alternatively, the occlusive element can be formed by dip coating the retention structure in a biocompatible polymer, for example silicone polymer. The thickness of the occlusive element can be in a range from about 0.01 mm to about 0.15 mm, and often from about 0.05 mm to 0.1 mm.

### Drug Core:

The drug core may be inserted into an implant body, or may serve as the implant itself, without any additional structural components. The drug core comprises latanoprost and materials to provide sustained release of the latanoprost. In some embodiments, the drug core comprises a sustained release formulation, which formulation consists of or consists essentially of latanoprost and silicone as a carrier. The latanoprost migrates from the drug core to the target tissue, for example ciliary muscles of the eye. The drug core may optionally comprise latanoprost in a matrix, wherein the latanoprost is dispersed or dissolved within the matrix. The latanoprost may be only slightly soluble in the matrix so that a small amount is dissolved in the matrix and available for release from the surface of the drug core. As the latanoprost diffuses from the exposed surface of the core to the tear or tear film, the rate of migration from the core to the tear or tear film can be related to the concentration of latanoprost dissolved in the matrix. In addition or in combination, the rate of migration of latanoprost from the core to the tear or tear film can be related to properties of the matrix in which the latanoprost is dissolved.

In an embodiment, the topical formulation or the drug core does not contain a preservative. Preservatives include, for example, benzalkonium chloride and EDTA. In an embodiment, the implants of the invention may be less allergenic and may reduce chemical sensitivity compared to formulations containing these preservatives.

In specific embodiments, the rate of migration from the drug core to the tear or tear film can be based on a silicone formulation. In some embodiments, the concentration of latanoprost dissolved in the drug core may be controlled to provide the desired rate of release of the latanoprost. The latanoprost included in the core can include liquid (such as oil), solid, solid gel, solid crystalline, solid amorphous, solid particulate, or dissolved forms of latanoprost. In a some embodiments, the drug core may comprise liquid or solid inclusions, for example liquid Latanoprost droplets dispersed in the silicone matrix.

Table 1 shows drug insert silicones that may be used and associated cure properties, according to embodiments of the present invention. The drug core insert matrix material can include a base polymer comprising dimethyl siloxane, such as MED-4011, MED 6385 and MED 6380, each of which is commercially available from NuSil. The base polymer can be cured with a cure system such as a platinum-vinyl hydride cure system or a tin-alkoxy cure system, both commercially available from NuSil. In many embodiments, the cure system may comprise a known cure system commercially available for a known material, for example a known platinum vinyl hydride cure system with known MED-4011. In a specific embodiment shown in Table 1, 90 parts of MED-4011 can be combined with 10 parts of the crosslinker, such that the crosslinker comprises 10% of the mixture. A mixture with MED-6385 may comprise 2.5% of the crosslinker, and mixtures of MED-6380 may comprise 2.5% or 5% of the crosslinker.

TABLE 1
Drug Insert Silicone Selections

| | | | Crosslinker |
|---|---|---|---|
| Material | Base Polymer | Cure System | Percent |
| MED-4011 | Dimethyl siloxane | Platinum vinyl | 10% |
| | Silica filler | hydride system | |
| | material | 10% | |
| MED-6385 | Dimethyl siloxane | Tin-Alkoxy 2.5% | 2.5% |
| | Diatomaceous | | |
| | earth filler material | | |
| MED-6380 | Dimethyl siloxane | Tin-Alkoxy | 2.5 to 5% |
| | without filler | | |
| | material | | |

It has been determined according to the present invention that the cure system and type of silicone material can affect the curing properties of the solid drug core insert, and may potentially affect the yield of therapeutic agent from the drug core matrix material. In specific embodiments, curing of MED-4011 with the platinum vinyl hydride system can be inhibited with high concentrations of drug/prodrug, for example over 20% drug, such that a solid drug core may not be formed. In specific embodiments, curing of MED-6385 or MED 6380 with the tin alkoxy system can be slightly inhibited with high concentrations, e.g. 20%, of drug/prodrug. This slight inhibition of curing can be compensated by increasing the time or temperature of the curing process. For example, embodiments of the present invention can make drug cores comprising 40% drug and 60% MED-6385 with the tin alkoxy system using appropriate cure times and temperatures. Similar results can be obtained with the MED-6380 system the tin-alkoxy system and an appropriate curing time or temperature. Even with the excellent results for the tin alkoxy cure system, it has been determined according to the present invention that there may be an upper limit, for example 50% drug/prodrug or more, at which the tin-alkoxy cure system may not produce a solid drug core. In many embodiments, the latanoprost in the solid drug core may be at least about 5%, for example a range from about 5% to 50%, and can be from about 20% to about 40% by weight of the drug core.

The drug core or other agent supply (e.g., implant impregnated body) can comprise one or more biocompatible materials capable of providing sustained release of latanoprost. Although the drug core is described above with respect to an embodiment comprising a matrix with a substantially non-biodegradable silicone matrix with inclusions of latanoprost located therein that dissolve, the drug core can include structures that provide sustained release of latanoprost, for example a biodegradable matrix, a porous drug core, liquid drug cores and solid drug cores.

A matrix that contains latanoprost can be formed from either biodegradable or non-biodegradable polymers. A non-biodegradable drug core can include silicone, acrylates, polyethylenes, polyurethane, polyurethane, hydrogel, polyester (e.g., DACRON.®. from E. I. Du Pont de Nemours and Company, Wilmington, Del.), polypropylene, polytetrafluoroethylene (PTFE), expanded PTFE (ePTFE), polyether ether ketone (PEEK), nylon, extruded collagen, polymer foam, silicone rubber, polyethylene terephthalate, ultra high molecular weight polyethylene, polycarbonate urethane, polyurethane, polyimides, stainless steel, nickel-titanium alloy (e.g., Nitinol), titanium, stainless steel, cobalt-chrome alloy (e.g., ELGILOY.®. from Elgin Specialty Metals, Elgin, Ill.; CONICHROME.®. from Carpenter Metals Corp., Wyomissing, Pa.).

A biodegradable drug core can comprise one or more biodegradable polymers, such as protein, hydrogel, polyglycolic acid (PGA), polylactic acid (PLA), poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid) and combinations thereof. In some embodiments the drug core can comprise at least one hydrogel polymer.

### Specific Implant Embodiments:

Various embodiments of the implant that may be employed in the methods described herein are as follows (see also the Example section below). In some embodiments, the drug insert includes a thin-walled polyimide tube sheath body that is filled with latanoprost dispersed in Nusil 6385, a cured medical grade solid silicone. The cured silicone serves as the solid, non-erodible matrix from which latanoprost slowly elutes. The drug insert is sealed at the distal end with a cured film of solid Loctite 4305 medical grade adhesive (cyanoacrylate). The polyimide tube sheath body is inert and, together with the adhesive, provides structural support and a barrier to both lateral drug diffusion and drug diffusion through the distal end of the drug insert. The drug insert is seated in the bore of the punctum plug and is held in place via an interference fit. In some embodiments, a body of the implant is at least partially impregnated with a therapeutic agent, such as latanoprost.

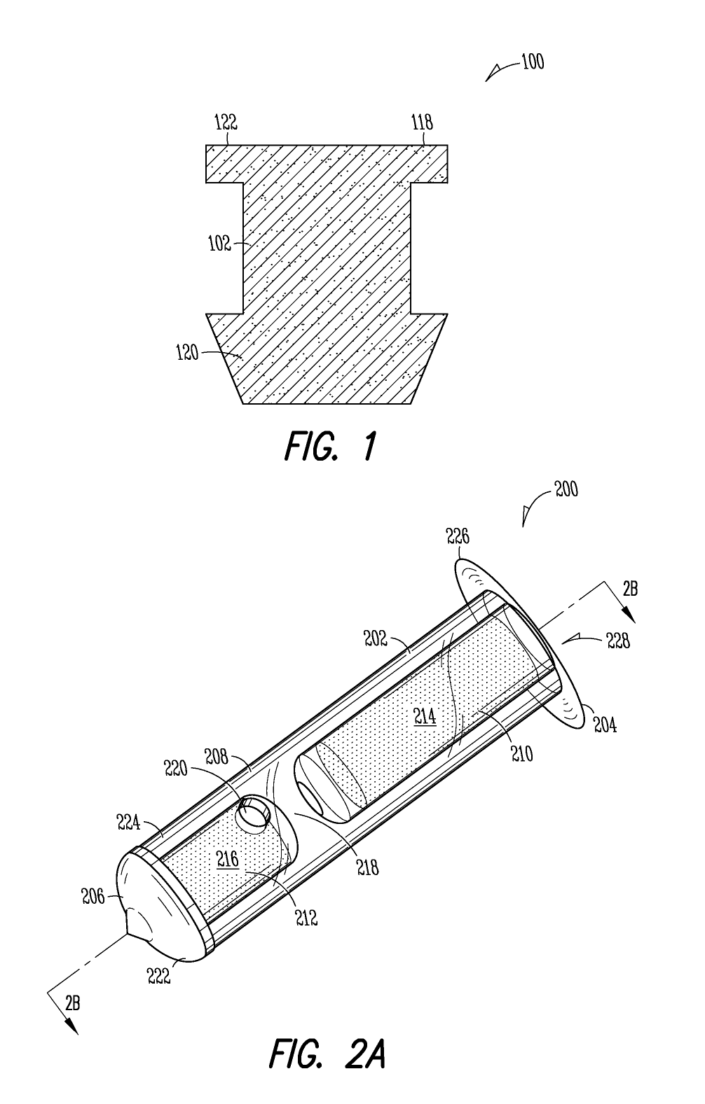

FIG. 1 illustrates an example embodiment of a cross-sectional view of a punctum plug 100 taken along a line parallel to a longitudinal axis of the plug. As shown in FIG. 1, the punctum plug 100 comprises a plug body 102. In the embodiment shown, the plug body 102 includes an integral feedback or other projection 122, such as a projection extending laterally at least partially from or around a proximal end 118 of the plug body 102. The projection 122 is in the form of a collarette extending radially outwardly from the plug body 102 to a degree sufficient so that at least a portion of the collarette will extend beyond and be exterior to the punctum after insertion of plug body 102 distal portions into the canaliculus.

In this embodiment, the plug body 102 is at least partially impregnated with a drug-releasing or other agent-releasing drug supply 120. In certain embodiments, the drug supply 120 is disposed within, dispersed throughout, or otherwise contained in the plug body 102. As discussed in commonly-owned Odrich, application Ser. No. 10/825,047 (filed Apr. 15, 200 and entitled Drug Delivery via Punctal Plug), which is herein incorporated by reference in its entirety, the agent of the drug supply 120 can be released from the plug body 102 into tear fluid of the eye or into the nasolacrimal duct system. In some embodiments, an impermeable sheath is disposed over portions of the plug body 102 to control drug supply 120 release therefrom.

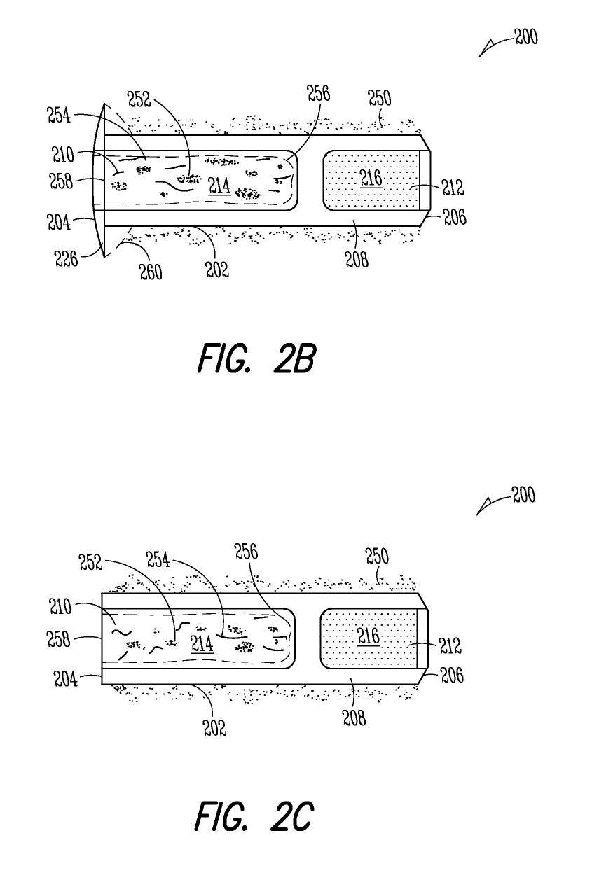

FIG. 2A illustrates an example embodiment of a punctum plug implant 200 that is insertable into a lacrimal punctum. The insertion of the punctum plug implant 200 into the lacrimal punctum allows for one or more of inhibition or blockage of tear flow therethrough (e.g., to treat dry eyes) or the sustained delivery of a therapeutic agent to an eye (e.g., to treat one or more of infection, inflammation, glaucoma or other ocular diseases). In this embodiment, the punctum plug 200 comprises a plug body 202 extending from a proximal end portion 204 to a distal end portion 206 and having a retention structure 208.

In various embodiments, the plug body 202 can comprise an elastic material, such as silicone, polyurethane or other urethane-based material, or an acrylic of a non-biodegradable, partially biodegradable or biodegradable nature (i.e., erodeable within the body) allowing at least one portion of the retention structure to deform outward. In some embodiments, the biodegradable elastic materials include cross-linked polymers, such as poly(vinyl alcohol). In some embodiments, different portions of the plug body 202 are made of different materials. For instance, the plug body proximal end portion 204 can comprise a silicone/polyurethane co-polymer and the plug body distal end portion 206 can comprise a polyurethane hydrogel or other solid hydrogel. In certain embodiments, the plug body proximal end portion 204 can comprise silicone and the plug body distal end portion 206 can comprise a hydrophilic silicone mixture. Other co-polymers that can be used to form the plug body 302 include silicone/urethane, silicone/poly(ethylene glycol) (PEG), and silicone/2hydroxyethyl methacrylate (HEMA).

In certain embodiments, the plug body 202 can include a cylindrical-like structure having a first chamber 210 at or near the proximal end and a second chamber 212 at or near the distal end. A latanoprost drug core 214 can be disposed in the first chamber 210, while a hydrogel or other expandable retention element 216 of a biodegradable or non-biodegradable nature can be disposed in the second chamber 216. In some embodiments, the biodegradable retention elements include salt and cellulose based mixtures. In some embodiments, the non-biodegradable retention elements include hydrogels or other synthetic polymers. A plug body septum 218 can be positioned between the first chamber 210 and the second chamber 216 and can be used to inhibit or prevent communication of a material between the drug core 214 and the hydrogel retention element 216.

In various ways, the expandable, hydrogel retention element 216 can be substantially encapsulated, such as within a portion of the retention structure 208. In various embodiments, the retention structure 208 can include a fluid permeable retainer allowing fluid to be received into and absorbed or otherwise retained by the hydrogel retention element 216, such as upon its insertion into the punctum. The hydrogel retention element 216 can be configured to expand, such as to a size or shape that urges one or more outer surface portions of the retention structure 208 to contact a wall of the lacrimal canaliculus, thereby retaining or helping retain a least a portion of the plug implant within the punctum. In some embodiments, the fluid permeable retainer can include a fluid permeable aperture 220, such as disposed in a lateral wall of the retention structure 208. In some embodiments, the fluid permeable retainer can include a fluid permeable or hydrophilic cap member 222 or other membrane. In some embodiments, the fluid permeable retainer can include a fluid permeable or hydrophilic plug body portion 224. These examples of fluid permeable retainers 220, 222, and 224 can also inhibit the hydrogel retention element 216 from appreciably protruding out of the retention structure 208 during and upon expansion.

The plug implant body 202 can include a feedback or other projection 226, such as extending laterally at least partially from or around (e.g., a removal loop) a proximal end portion 204 of the plug body 202. In some embodiments, the projection 226 can include a removal loop. In some embodiments, the projection 226 can be configured to seat against or near (e.g., via a ramped portion 260) the punctum opening, such as for inhibiting or preventing the punctum plug 200 from passing completely within the canaliculus, or for providing tactile or visual feedback information to an implanting user regarding the same. In some embodiments, a proximal end of the projection 226 can include a convex such as for helping provide comfort to a patient when implanted. In some embodiments, the projection 226 can include a convex radius of about 0.8 millimeters. In some embodiments, the projection 226 is between about 0.7 millimeters to about 0.9 millimeters in diameter. In some embodiments, the projection 226 can include a non-concave shape of about 0.5 millimeters to about 1.5 millimeters in diameter, and 0.1 millimeters to about 0.75 millimeters in thickness. In some embodiments, the projection 226 has a wing-like shape, in which a column-like projection extends from opposite sides of the implant plug proximal end 204. In some examples, the projection 226 includes a partially trimmed collar extending 360 degrees around the proximal end 204 from an outer plug body surface. In some examples, such the projection 226 includes a full collar extending 360 degrees around the proximal end 204 from an outer plug body surface. In an example, the projection 226 includes a cross-sectional shape similar to a flat disk (i.e., relatively flat top and bottom surfaces). A drug or other agent elution port 228 can extend though the projection 226, such as to provide sustained release of a drug core 214 agent onto an eye.

FIG. 2B illustrates a cross-sectional view of an example embodiment of a punctum plug implant 200 taken along a line parallel to a longitudinal axis of the implant, such as along line 2B-2B of FIG. 2A. As shown in FIG. 2B, the punctum plug can include a plug body 202 having a retention structure 208 substantially encapsulating a hydrogel retention element 216 at or near a plug body distal end portion 206, and a latanoprost drug core 214 disposed within the plug body, for example at or near a proximal end portion 204. In this embodiment, the drug core 214 is disposed in a first plug body chamber 210 and the hydrogel retention element 216 is disposed in a second plug body chamber 212. As discussed above, the hydrogel retention element 216 can be configured to expand to a size or shape that retains or helps retain at least a portion of the plug implant 200 within the lacrimal punctum. In some embodiments, a hydrogel retention element 250 can also be coated or otherwise provided on an outer surface portion of the plug body 202 providing another (e.g., secondary) mechanism for retaining or helping to retain at least a portion of the plug 200 at least partially within the lacrimal punctum.

The retention structure 208, which can be used to substantially encapsulate the hydrogel retention element 216, can be of varying sizes relative to a plug body 202 size. In some embodiments, the retention structure 208 is at least about one fifth the length of the plug body 202. In some embodiments, the retention structure 208 is at least about one fourth the length of the plug body 202. In some embodiments, the retention structure 208 is at least about one third the length of the plug body 202. In some embodiments, the retention structure 208 is at least about one half the length of the plug body 202. In some embodiments, the retention structure 208 is at least about three quarters the length of the plug body 202. In some embodiments, the retention structure 208 is about the full length of the plug body 202.

As shown in the example embodiment of FIG. 2B, the hydrogel retention element 216 can have a non-expanded, “dry” state, which aids insertion through the punctum and into the lacrimal canaliculus. Once placed in the canaliculus, the hydrogel retention element 216 can absorb or otherwise retain canalicular or other fluid, such as via a fluid permeable retainer 220, 222, 224 (FIG. 2A) to form an expanded structure. In some embodiments, the hydrogel retention element 216 can include a material that is non-biodegradable. In some embodiments, the hydrogel retention element 216 can include a material that is biodegradable. Other options for the hydrogel retention element 216 can also be used. For instance, the hydrogel retention element 216 can be molded with the retention structure 208 in a single piece, or can be formed separately as one piece and subsequently coupled to the retention structure 208.

In some examples, the drug core 214 disposed at or near the proximal end portion 204 of the plug body 202 can include a plurality of latanoprost inclusions 252, which can be distributed in a matrix 254. In some embodiments, the inclusions 252 comprise a concentrated form of the latanoprost (e.g., a crystalline agent form). In some embodiments, the matrix 254 can comprise a silicone matrix or the like, and the distribution of inclusions 252 within the matrix can be non-homogeneous. In some embodiments, the agent inclusions 252 include droplets of an oil, such as latanoprost oil. In still other embodiments, the agent inclusions 252 comprise solid particles. The inclusions can be of many sizes and shapes. For instance, the inclusions can be microparticles having dimensions on the order of about 1 micrometers to about 100 micrometers.

In the embodiment shown, the drug core 214 has a sheath body 256 disposed over at least a portion thereof such as to define at least one exposed surface 258 of the drug core. The exposed surface 258 can be located at or near the proximal end portion 204 of the plug body such as to contact a tear or a tear film fluid and release the latanoprost at one or more therapeutic levels over a sustained time period when the punctum plug 200 is inserted into the punctum.

FIG. 2C illustrates a cross-sectional view of an example embodiment of a punctum plug 200 taken along a line parallel to a longitudinal axis of the plug. As shown in FIG. 2C, the punctum plug includes a plug body 202 without a feedback or other projection 226 (FIG. 2A). In this way, the plug 200 can be completely inserted inside the lacrimal punctum. In some embodiments, the first chamber 210 can include dimensions of about 0.013 inches×about 0.045 inches. In some embodiments, the second chamber 212 can include dimensions of about 0.013 inches by about 0.020 inches.

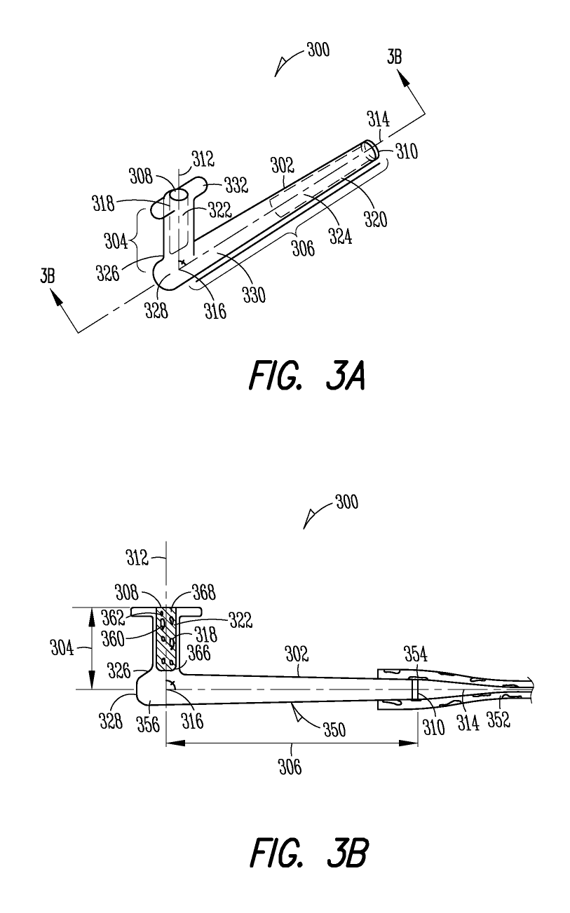

FIG. 3A illustrates another embodiment of a punctum plug implant 300 that can be insertable into a lacrimal punctum. The insertion of the punctum plug 300 into the lacrimal punctum can allow for one or more of: inhibition or blockage of tear flow therethrough (e.g., to treat dry eyes) or the sustained delivery of a therapeutic agent to an eye (e.g., to treat an infection, inflammation, glaucoma or other ocular disease or disorder), a nasal passage (e.g., to treat a sinus or allergy disorder) or an inner ear system (e.g., to treat dizziness or a migraine).

In this embodiment, the punctum plug 300 comprises a plug body 302 including first 304 and second 306 portions. The plug body 302 extends from a proximal end 308 of the first portion 304 to a distal end 310 of the second portion 306. In various embodiments, the proximal end 308 can define a longitudinal proximal axis 312 and the distal end 310 can define a longitudinal distal axis 314. The plug body 300 can be configured such that, when implanted, an at least 45 degree angled intersection 316 exists between the proximal axis 312 and the distal axis 314 for biasing at least a portion of the plug body 302 against at least a portion of a lacrimal canaliculus located at or more distal to a canaliculus curvature. In some embodiments, the plug body 302 can be configured such that the angled intersection 316 is between about 45 degrees and about 135 degrees. In this embodiment, the plug body 302 is configured such that the angled intersection 316 is approximately about 90 degrees. In various embodiments, a distal end 326 of the first portion 304 can be integral with the second portion 306 at or near a proximal end 328 of the second portion 306.

In certain embodiments, the plug body 302 can include angularly disposed cylindrical-like structures comprising one or both of a first cavity 318 disposed near the proximal end 308 or a second cavity 320 disposed near the distal end 310. In this embodiment, the first cavity 318 extends inward from the proximal end 308 of the first portion 304, and the second cavity 320 extends inward from the distal end 310 of the second portion 306. A first drug-releasing drug supply 322 can be disposed in the first cavity 318 to provide a sustained drug release to an eye, while a second drug-releasing or other agent-releasing drug supply 324 can be disposed in the second cavity 320 to provide a sustained drug or other agent release to a nasal passage or inner ear system, for example. A plug body septum 330 can be positioned between the first cavity 318 and the second cavity 320, and can be used to inhibit or prevent communication of a material between the first drug supply 322 and the second drug supply 324.

In some embodiments, the drug or other agent release can occur, at least in part, via an exposed surface of the drug supply 322, 324. In some embodiments, by controlling geometry of the exposed surface, a predetermined drug or agent release rate can be achieved. For instance, the exposed surface can be constructed with a specific geometry or other technique appropriate to control the release rate of the drug or other agent onto an eye, such as on an acute basis, or on a chronic basis between outpatient doctor visits, for example. Further description regarding effective release rates of one or more drugs or other agents from a drug supply 322, 324 can be found in commonly-owned DeJuan et al., U.S. application Ser. No. 11/695,545 (filed Apr. 2, 2007 and entitled Nasolacrimal Drainage System Implants for Drug Therapy) which is herein incorporated by reference in its entirety, including its description of obtaining particular release rates. In some embodiments, the exposed surface of the drug supply 322, 324 can be flush or slightly below the proximal end 308 of the first portion 304 or the distal end 310 of the second portion 306, respectively, such that the drug supply does not protrude outside of the plug body 302. In some embodiments, the exposed surface of the drug supply 322, for instance, can be positioned above the proximal end 308 such that the drug supply 322 at least partially protrudes outside of the plug body 302.

The plug body 302 can include an integral feedback or other projection 332, such as projections extending laterally at least partially from or around a proximal end 308 of the first plug body portion 304. In some embodiments, the projection 332 can include a set of wings for use in removing the punctum plug 300 from an implant position. The removal set of wings can be configured without migration in mind, as the non-linear configuration of the plug body 302 can prevent migration by assuming a size or shape of the canaliculus curvature and optionally, the lacrimal canaliculus ampulla. In some embodiments, the projection 332 can be configured to seat against or near the punctal opening such as for inhibiting or preventing the punctum plug 300 from passing completely within the lacrimal canaliculus, or for providing tactile or visual feedback information to an implanting user, e.g., as to whether the plug is fully implanted. The projection 332 can extend laterally in a direction parallel to or away from an eye when implanted. This will reduce irritation to the eye as compared to a case in which a portion of the projection extends toward the eye. In addition, a lateral extension direction of the projection 332 from the proximal end 308 can be substantially the same as a lateral extension direction of the second plug body portion 306 relative to the distal end 326 of the first plug body portion 304. This can also avoid extension toward the eye. A drug or other agent elution port can extend though a collar-projection 332, such as to provide sustained release of the drug supply 322 agent onto an eye.

In various embodiments, the plug body 302 can be molded using an elastic material, such as silicone, polyurethane, NuSil (e.g., NuSil 4840 with 2% 6-4800) or an acrylic of a non-biodegradable, partially biodegradable or biodegradable nature (i.e., erodeable within the body) allowing a non-linear extending plug body 302 to be formed. In some embodiments, the biodegradable elastic materials can include cross-linked polymers, such as poly(vinyl alcohol). In some embodiments, the plug body 302 can comprise a silicone/polyurethane co-polymer. Other co-polymers that can be used to form the plug body 302 include, but are not limited to, silicone/urethane, silicone/poly(ethylene glycol) (PEG), and silicone/2hydroxyethyl methacrylate (HEMA). As discussed in commonly-owned Jain et al., Application Ser. No. 61/049,317 (filed Apr. 30, 2008 and entitled Drug-Releasing Polyurethane Lacrimal Insert), which is herein incorporated by reference in its entirety, urethane-based polymer and copolymer materials allow for a variety of processing methods and bond well to one another.

FIG. 3B illustrates an example embodiment of a cross-sectional view of a punctum plug 300 taken along a line parallel to a longitudinal axis of the plug, such as along line 3B-3B of FIG. 3A. As shown in FIG. 3B, the punctum plug 300 can include a plug body 302 including first 304 and second 306 portions. The plug body 302 extends from a proximal end 308 of the first portion 304 to a distal end 310 of the second portion 306. In various embodiments, the proximal end 308 can defines a longitudinal proximal axis 312 and the distal end 310 can define a longitudinal distal axis 314. The plug body 300 can be configured such that, when implanted, an at least 45 degree angled intersection 316 exists between the proximal axis 312 and the distal axis 314 for biasing at least a portion of the plug body 302 against at least a portion of a lacrimal canaliculus located at or more distal to a canaliculus curvature. In this embodiment, the plug body 300 is configured such that the angled intersection 316 is approximately about 90 degrees.

In various embodiments, a distal end 326 of the first portion 304 can be integral with the second portion 306 at or near a proximal end 328 of the second end 326. In some embodiments, the second portion 306 can include a length having a magnitude less than four times a length of the first portion 304. In one embodiment, the second portion 306 can include a length of less than about 10 millimeters, such as is shown in FIG. 3B. In another embodiment, the second portion 306 can include a length less than about 2 millimeters.

In certain embodiments, the second portion 306 can comprise an integral dilator 350 to dilate anatomical tissue 352, such one or both of a lacrimal punctum or canaliculus to a sufficient diameter as the punctum plug 300 is being implanted. In this way, the punctum plug 300 can be implanted in various size ocular anatomies without the need for pre-dilation via a separate enlarging tool. The dilator 350 can be formed so as to not be traumatic to an inner lining of the punctum and the canaliculus. In some embodiments, a lubricious coating disposed on, or impregnated in, an outer surface of the plug body 302 can be used to further aid insertion of the punctum plug 300 into the anatomical tissue 352. In one embodiment, the lubricious coating can include a silicone lubricant.

As shown, the dilator 350 can generally narrow from a location near the proximal end 328 of the second portion 306 to the distal end 310 of the second portion 306, such as from a diameter of about 0.6 millimeters to a diameter of about 0.2 millimeters. In some embodiments, an outer surface slope of the dilator 350, as measured from the location near the proximal end 328 of the second portion 306 to the distal end 310 of the second portion 306, can be between about 1 degree and about 10 degrees (e.g., 2 degrees, 3 degrees, 4 degrees, or 5 degrees) with respect to the longitudinal distal axis 314. In some embodiments, the slope of the dilator 350 can be less than 45 degrees with respect to the longitudinal distal axis 314. Among other factors, a determination of a desirable dilator 350 slope for a given implant situation can be made by balancing a plug body 302 strength desirable for plug implant with a desire to have a soft, flexible and conforming plug body (e.g., to conform to a lacrimal canaliculus anatomy) upon implantation. In some embodiments, a diameter of a dilator tip 354 can be between about 0.2 millimeters and about 0.5 millimeters.

In certain embodiments, the proximal end 328 of the second plug body portion 306 can include a lead extension 356 configured to bias against at least a portion of a lacrimal canaliculus ampulla when implanted. In this embodiment, the lead extension 356 projects proximally from the intersection between the first 304 and second 306 plug body portions, such as in an opposite direction as the extension of the dilator 350.

In certain embodiments, the plug body 302 can include a first cavity 318 disposed near the proximal end 308. In this embodiment, the first cavity 318 extends inward about 2 millimeters or less from the proximal end 308, and houses a first drug-releasing or other agent-releasing drug supply 322 to provide a sustained drug or other agent release to an eye. In some embodiments, the drug supply 322 can include a plurality of therapeutic agent inclusions 360, which can be distributed in a matrix 362. In some embodiments, the inclusions 360 can comprise a concentrated form of the therapeutic agent (e.g., a crystalline agent form). In some embodiments, the matrix 362 can comprise a silicone matrix or the like, and the distribution of inclusions 360 within the matrix can be non-homogeneous. In some embodiments, the agent inclusions 360 can include droplets of oil, such as latanoprost oil. In still other embodiments, the agent inclusions 360 can comprise solid particles, such as Bimatoprost particles in crystalline form. The inclusions can be of many sizes and shapes. For instance, the inclusions can include microparticles having dimensions on the order of about 1 micrometer to about 100 micrometers.

In the embodiment shown, the drug supply 322 includes a sheath body 366 disposed over at least a portion thereof such as to define at least one exposed surface 368 of the drug supply. The exposed surface 368 can be located at or near the proximal end 308 of the plug body 302 such as to contact a tear or a tear film fluid and release the therapeutic agent at one or more therapeutic levels over a sustained time period when the punctum plug 300 is inserted into the lacrimal punctum.

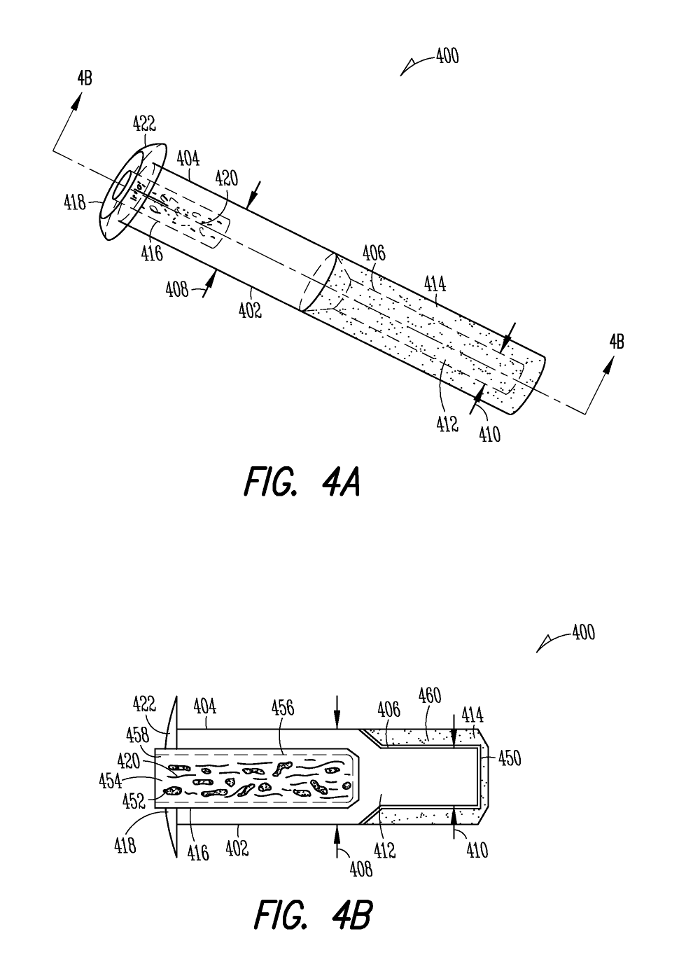

FIG. 4A illustrates an embodiment of a punctum plug 400 that can be insertable into a lacrimal punctum. In various embodiments, the punctum plug 400 comprises a plug body 402, including first 404 and second 406 portions, which is sized and shaped for at least partial insertion into a lacrimal punctum. The first portion 404 is formed from a polymer and includes a first diameter 408. The second portion 406 is also formed from a polymer and includes a base member 412 (e.g., mandrel or spine-like member) having a second diameter 410, which is less than the first diameter 408. In an embodiment, the first 404 and second 406 portions are integrally coupled and comprise a unitary plug body 402. In an embodiment, the first 404 and second 406 portions are separate elements, which can be coupled to one another via an engagement between a coupling void and a coupling arm, for instance.

An expandable retention member 414, such as a swellable material, can be bonded or otherwise coupled over the base member 412 such that it envelops, at least in part, a portion of the base member 412. In an embodiment, the expandable retention member substantially envelops the base member 412. As the expandable retention member 414 absorbs or otherwise retains lacrimal or other fluid, such as upon insertion into a lacrimal punctum, its size increases and its shape may change thereby urging itself against and slightly biasing a wall of the associated canaliculus. It is believed that the expandable retention member 414 will provide retention comfort to a subject and may improve punctum plug 400 implant retention via controlled biasing of the canaliculus wall.

The positioning of the expandable retention member 414 over a portion of the plug body 402 allows the retention member 414 to be freely exposed to lacrimal fluid in situ, thereby allowing for a wide range of potential expansion rates. Further, the base member 412 provides an adequate coupling surface area to which the expandable retention member 414, for example, can adhere such that the material of the expandable retention member 414 does not remain in a lacrimal punctum after the punctum plug 400 is removed from the subject. As shown in this embodiment, the expandable retention member 414 can include a non-expanded, “dry or dehydrated” state, which aids insertion through a lacrimal punctum and into the associated lacrimal canaliculus. Once placed into a lacrimal canaliculus, the expandable retention member 414 can absorb or other retain lacrimal fluid to form an expanded structure.

In some embodiments, the plug body 402 can include a cylindrical-like structure comprising a cavity 416 disposed near a proximal end 418 of the first portion 404. In this embodiment, the cavity 416 extends inward from the proximal end 418 and includes a first drug-releasing or other agent-releasing drug supply 420 to provide a sustained drug or other agent release to an eye. The drug or other agent release can occur, at least in part, via an exposed surface of the drug supply 420. In an embodiment, the exposed surface of the drug supply 420 can be positioned above the proximal end 418 such that the drug supply 420 at least partially protrudes outside of the plug body 402. In some embodiments, the exposed surface of the drug supply 420 can be flush or slightly below the proximal end 418 such that the drug supply 420 does not protrude outside of the plug body 402.

In some embodiments, by controlling geometry or a drug concentration gradient near the exposed surface, a predetermined drug or agent release rate can be achieved. For instance, the exposed surface can be constructed with a specific geometry or other technique appropriate to control the release rate of the drug or other agent onto an eye, such as on an acute basis, or on a chronic basis between outpatient doctor visits, for example.

The plug body 402 can include an integral feedback or other projection 422, such as projections extending laterally at least partially from or around the proximal end 418 of the first plug body portion 404. In an embodiment, the projection 422 includes a partially trimmed collar extending 360 degrees around the proximal end 418 from an outer plug body surface. In an embodiment, the projection 422 includes a full collar extending 360 degrees around the proximal end 418 from an outer plug body surface. In an embodiment, the projection 422 includes a cross-sectional shape similar to a flat disk (i.e., relatively flat top and bottom surfaces). In various embodiments, the projection 422 can be configured to seat against or near a punctal opening when the second portion 406 of the plug body 402 is positioned within the associated canalicular lumen, such as for inhibiting or preventing the punctum plug 400 from passing completely within the canalicular lumen, for providing tactile or visual feedback information to an implanting user (e.g., as to whether the plug is fully implanted), or for removing the punctum plug 400 from an implant position. In an embodiment, the projection 422 includes a portion having a diameter of about 0.5-2.0 mm to prevent the punctum plug 400 from passing down into the canaliculus.

FIG. 4B illustrates an example embodiment of a cross-sectional view of a punctum plug 400 taken along a line parallel to a longitudinal axis of the plug, such as along line 4B-4B of FIG. 4A. As shown in FIG. 4B, the punctum plug 400 comprises a plug body 402, including first 404 and second 406 portions, which is sized and shaped for at least partial insertion into a lacrimal punctum. The first portion 404 is formed from a polymer and includes a first diameter 408. The second portion 406 is also formed from a polymer and includes a base member 412 (e.g., mandrel or spine) having a second diameter 410, which is less than the first diameter 408. In an embodiment, the base member 412 is at least about one-third the total length of the plug body 402. In an embodiment, the base member 412 is at least about one-half the total length of the plug body 402. In the embodiment shown, the plug body 402 also includes an integral feedback or other projection 422, such as a projection extending laterally at least partially from or around a proximal end 418 of the first plug body portion 404.

In various embodiments, the plug body 402 can be molded or otherwise formed using an elastic material, such as silicone, polyurethane or other urethane-based material, or combinations thereof. In an embodiment, one or both of the first 404 and second 406 portions include a urethane-based material. In an embodiment, one or both of the first 404 and second 406 portions include a silicone-based material, such as 4840® or PurSil®. PurSil® is further described in U.S. Pat. Nos. 5,589,563 and 5,428,123, the disclosures of which are incorporated herein by reference in their entirety. In an embodiment, one or both of the first 404 and second 406 portions include a copolymer material, such as polyurethane/silicone, urethane/carbonate, silicone/polyethylene glycol (PEG) or silicone/2hydroxyethyl methacrylate (HEMA). In various embodiments, the plug body 402 is configured to be non-absorbable in situ and is sufficiently strong to address issues of cutting strength (e.g., during insertion and removal of the punctum plug 400) and dimensional stability.

An expandable retention member 414, such as a swellable material, can be bonded or otherwise coupled over the base member 412 such that it envelops, at least in part, a portion of the base member 412. As the expandable retention member absorbs or otherwise retains lacrimal fluid, such as upon insertion into a lacrimal punctum, its size increases and its shape may change thereby urging itself against and slightly biasing a wall of the associated canaliculus. In various embodiments, the expandable retention member 414 can be molded or otherwise formed using a swellable material. In an embodiment, the expandable retention member 414 includes a polyurethane hydrogel, such as TG-2000®, TG-500®, or other urethane-based hydrogel. In an embodiment, the expandable retention member 414 includes a thermoset polymer, which may be configured to swell anisotropically. In an embodiment, the expandable retention member 414 includes a gel, which does not maintain its shape upon expansion, but rather conforms to fit the shape of a canaliculus lumen wall or other surrounding structure.

In some embodiments, the punctum plug 400 includes a base member 412 including polyurethane or other urethane-based material and an expandable retention member 414 including a polyurethane or other urethane-based swellable material. In an embodiments, a polyurethane hydrogel is coupled directly to an outer surface, such as a plasma-treated outer surface, of the base member 412.

In some embodiments, the punctum plug 400 includes an intermediate member 450 positioned between a portion of the plug body 402, such as the base member 412, and a portion of the expandable retention member 414. The intermediate member 450 can include a material configured to absorb, when implanted, a greater amount of lacrimal fluid than the polymer of the base member 412 but less lacrimal fluid than the swellable polymer of the expandable retention member 414. The intermediate member 450 can provide the punctum plug 400 with integrity, such as between a substantially non-swelling polymer of the plug body 402 and a swelling polymer of the expandable retention member 414. For instance, when the polymer of the expandable retention member 414 swells upon exposure to moisture, it is possible that the expanding polymer will, in the absence of the intermediate member 450, swell away from the underlying, non-swelling polymer of the base member 412. In an embodiment, the intermediate member 450 includes PurSil® and is dip or otherwise coated onto an outer surface of the base member 412. In an embodiment, the intermediate member 450 includes a polyurethane configured to absorb about 10% to about 500% water, such as Tecophilic® urethanes or Tecophilic® solution grade urethanes. Further discussion regarding the use of an intermediate member 450 positioned between a portion of a first polymer material and a portion of a second polymer material, typically different than the first polymer material, can be found in commonly-owned Sim et al., U.S. Application Ser. No. 61/049,329 (filed Apr. 30, 2008 and entitled Composite Lacrimal Insert), which is herein incorporated by reference in its entirety.

In certain embodiments, the plug body 402 can include a cavity 416 disposed near the proximal end 418 of the first portion 404. In an embodiment, the first cavity 416 extends inward about 2 millimeters or less from the proximal end 418, and houses a first drug-releasing or other agent-releasing drug supply 420 to provide a sustained drug or other agent release to an eye. In an embodiment, the first cavity 416 extends through the plug body 402, and houses a first drug-releasing or other agent-releasing drug supply 420. In various embodiments, the drug supply 420 stores and slowly dispenses an agent to one or both of the eye or the nasolacrimal system as they are leached out, for example, by tear film fluid or other lacrimal fluid. In an embodiment, the drug supply 420 includes a plurality of therapeutic agent inclusions 452, which can be distributed in a matrix 454. In an embodiment, the inclusions 452 comprise a concentrated form of the therapeutic agent (e.g., a crystalline agent form). In an embodiment, the matrix 454 comprises a silicone matrix or the like, and the distribution of inclusions 452 within the matrix are homogeneous or non-homogeneous. In an embodiment, the agent inclusions 452 include droplets of oil, such as Latanoprost oil. In still another embodiment, the agent inclusions 452 include solid particles, such as Bimatoprost particles in crystalline form. The inclusions can be of many sizes and shapes. For instance, the inclusions can include microparticles having dimensions on the order of about 1 micrometer to about 100 micrometers.

In the embodiment shown, the drug supply 420 includes a sheath body 456 disposed over at least a portion thereof such as to define at least one exposed surface 458 of the drug supply. In an embodiment, the sheath body 456 comprises polyimide. The exposed surface 458 can be located at or near the proximal end 418 of the plug body 402 such as to contact a tear or a tear film fluid and release the therapeutic agent at one or more therapeutic levels over a sustained time period when the punctum plug 400 is inserted into a lacrimal punctum.

In certain embodiments, the expandable retention member can include a second drug-releasing or other agent-releasing drug supply 460 to provide a sustained drug or other agent release to one or both of a wall of a lacrimal canaliculus or a nasolacrimal system. The drug supply 460 can be configured to store and slowly dispense an agent after contact with lacrimal fluid within a lacrimal canaliculus. In an embodiment, the agent included in the expandable retention member can comprise medicaments, therapeutic agents, or antimicrobials (e.g., silver).

### Making the Implant:

Those of skill in the art will be familiar with various methods useful for making the implants described herein. Particular methods are described in the above-identified patent documents, the disclosures of which are incorporated herein by reference in their entirety.

For example, drug cores as described above may be fabricated with different cross sectional sizes of 0.006 inches, 0.012 inches, and 0.025 inches. Drug concentrations in the core may be 5%, 10%, 20%, 30% in a silicone matrix. These drug cores can be made with a syringe tube and cartridge assembly, mixing latanoprost with silicone, and injecting the mixture into a polyimide tube which is cut to desired lengths and sealed. The length of the drug cores can be approximately 0.80 to 0.95 mm, which for a diameter of 0.012 inches (0.32 mm) corresponds to total latanoprost content in the drug cores of approximately 3.5 micrograms, 7 micrograms, 14 micrograms and 21 micrograms for concentrations of 5%, 10%, 20% and 30%, respectively.

Syringe Tube and Cartridge Assembly: 1. Polyimide tubing of various diameters (for example 0.006 inches, 0.0125 inches and 0.025 inches) can be cut to 15 cm length. 2. The polyimide tubes can be inserted into a Syringe Adapter. 3. The polyimide tube can be adhesive bonded into luer adapter (Loctite, low viscosity UV cure). 4. The end of the assembly can then be trimmed. 5. The cartridge assembly can be cleaned using distilled water and then with methanol and dried in oven at 60.degree. C.

The latanoprost can be mixed with silicone. Latanoprost may be provided as a 1% solution in methylacetate. The appropriate amount of solution can be placed into a dish and using a nitrogen stream, the solution can be evaporated until only the latanoprost remains. The dish with the latanoprost oil can be placed under vacuum for 30 minutes. This latanoprost can then be combined with silicone, with three different concentrations of latanoprost (5%, 10% and 20%) in silicone Nusil 6385 being injected into tubing of different diameters (0.006 in, 0.012 in and 0.025 inches) to generate 3×3 matrixes. The percent of latanoprost to silicone is determined by the total weight of the drug matrix. Calculation: Weight of latanoprost/(weight of latanoprost+weight of silicone)×100=percent drug.

The tube can then be injected: 1. The cartridge and polyimide tubes assembly can be inserted into a 1 ml syringe. 2. One drop of catalyst (MED-6385 Curing Agent) can be added in the syringe. 3. Excess catalyst can be forced out of the polyimide tube with clean air. 4. The syringe can then be filled with silicone drug matrix. 5. The tube can then be injected with drug matrix until the tube is filled or the syringe plunger becomes too difficult to push. 6. The distal end of the polyimide tube can be closed off and pressure can be maintained until the silicone begins to solidify. 7. Allow to cure at room temperature for 12 hours. 8. Place under vacuum for 30 minutes. 9. The tube can then be place in the correct size trim fixture (prepared in house to hold different size tubing) and drug inserts can be cut to length (0.80-0.95 mm).

Release of Latanoprost from Punctum Plug:

The rate of release of latanoprost can be related to the concentration of latanoprost dissolved in the drug core. In some embodiments, the drug core comprises non-therapeutic agents that are selected to provide a desired solubility of the latanoprost in the drug core. The non-therapeutic agent of the drug core can comprise polymers as described herein, and additives. A polymer of the core can be selected to provide the desired solubility of the latanoprost in the matrix. For example, the core can comprise hydrogel that may promote solubility of hydrophilic treatment agent. In some embodiments, functional groups can be added to the polymer to provide the desired solubility of the latanoprost in the matrix. For example, functional groups can be attached to silicone polymer.

Additives may be used to control the concentration of latanoprost by increasing or decreasing solubility of the latanoprost in the drug core so as to control the release kinetics of the latanoprost. The solubility may be controlled by providing appropriate molecules or substances that increase or decrease the content of latanoprost in the matrix. The latanoprost content may be related to the hydrophobic or hydrophilic properties of the matrix and latanoprost. For example, surfactants and salts can be added to the matrix and may increase the content of hydrophobic latanoprost in the matrix. In addition, oils and hydrophobic molecules can be added to the matrix and may increase the solubility of hydrophobic treatment agent in the matrix.

Instead of or in addition to controlling the rate of migration based on the concentration of latanoprost dissolved in the matrix, the surface area of the drug core can also be controlled to attain the desired rate of drug migration from the core to the target site. For example, a larger exposed surface area of the core will increase the rate of migration of the treatment agent from the drug core to the target site, and a smaller exposed surface area of the drug core will decrease the rate of migration of the latanoprost from the drug core to the target site. The exposed surface area of the drug core can be increased in any number of ways, for example by any of castellation of the exposed surface, a porous surface having exposed channels connected with the tear or tear film, indentation of the exposed surface, protrusion of the exposed surface. The exposed surface can be made porous by the addition of salts that dissolve and leave a porous cavity once the salt dissolves. Hydrogels may also be used, and can swell in size to provide a larger exposed surface area. Such hydrogels can also be made porous to further increase the rate of migration of the latanoprost.

Further, an implant may be used that includes the ability to release two or more drugs in combination, such as the structure disclosed in U.S. Pat. No. 4,281,654 (Shell). For example, in the case of glaucoma treatment, it may be desirable to treat a patient with multiple prostaglandins or a prostaglandin and a cholinergic agent or an adrenergic antagonist (beta blocker), such as Alphagan.®., or latanoprost and a carbonic anhydrase inhibitor.

In addition, drug impregnated meshes may be used such as those disclosed in US Patent Publication No. 2002/0055701 (Ser. No. 77/2693) or layering of biostable polymers as described in US Patent Publication No. 2005/0129731 (Ser. No. 97/9977), the disclosures of which are incorporated herein in their entirety. Certain polymer processes may be used to incorporate latanoprost into the devices of the present invention; such as so-called “self-delivering drugs” or PolymerDrugs (Polymerix Corporation, Piscataway, N.J.) are designed to degrade only into therapeutically useful compounds and physiologically inert linker molecules, further detailed in US Patent Publication No. 2005/0048121 (Ser. No. 86/1881; East), hereby incorporated by reference in its entirety. Such delivery polymers may be employed in the devices of the present invention to provide a release rate that is equal to the rate of polymer erosion and degradation and is constant throughout the course of therapy. Such delivery polymers may be used as device coatings or in the form of microspheres for a drug depot injectable (such as a reservoir of the present invention). A further polymer delivery technology may also be configured to the devices of the present invention such as that described in US Patent Publication No. 2004/0170685 (Ser. No. 78/8747; Carpenter), and technologies available from Medivas (San Diego, Calif.).

In specific embodiments, the drug core matrix comprises a solid material, for example silicone, that encapsulates inclusions of the latanoprost. The drug comprises molecules which are very insoluble in water and slightly soluble in the encapsulating drug core matrix. The inclusions encapsulated by the drug core can be micro-particles having dimensions from about 1 micrometer to about 100 micrometers across. The drug inclusions can comprise droplets of oil, for example latanoprost oil. The drug inclusions can dissolve into the solid drug core matrix and substantially saturate the drug core matrix with the drug, for example dissolution of latanoprost oil into the solid drug core matrix. The drug dissolved in the drug core matrix is transported, often by diffusion, from the exposed surface of the drug core into the tear film. As the drug core is substantially saturated with the drug, in many embodiments the rate limiting step of drug delivery is transport of the drug from the surface of the drug core matrix exposed to the tear film. As the drug core matrix is substantially saturated with the drug, gradients in drug concentration within the matrix are minimal and do not contribute significantly to the rate of drug delivery. As surface area of the drug core exposed to the tear film is nearly constant, the rate of drug transport from the drug core into the tear film can be substantially constant. It has been determined according to the present invention that the solubility of the latanoprost in water and molecular weight of the drug can affect transport of the drug from the solid matrix to the tear. In many embodiments, the latanoprost is nearly insoluble in water and has a solubility in water of about 0.03% to 0.002% by weight and a molecular weight from about 400 grams/mol. to about 1200 grams/mol.

In many embodiments the latanoprost has a very low solubility in water, for example from about 0.03% by weight to about 0.002% by weight, a molecular weight from about 400 grams per mole (g/mol) to about 1200 g/mol, and is readily soluble in an organic solvent. Latanoprost is a liquid oil at room temperature, and has an aqueous solubility of 50 micrograms/mL in water at 25 degrees C., or about 0.005% by weight and a M.W. of 432.6 g/mol.

It has been determined according to the present invention that naturally occurring surfactants in the tear film, for example surfactant D and phospholipids, may effect transport of the drug dissolved in the solid matrix from the core to the tear film. The drug core can be configured in response to the surfactant in the tear film to provide sustained delivery of latanoprost into the tear film at therapeutic levels. For example, empirical data can be generated from a patient population, for example 10 patients whose tears are collected and analyzed for surfactant content. Elution profiles in the collected tears for a drug that is sparingly soluble in water can also be measured and compared with elution profiles in buffer and surfactant such that an in vitro model of tear surfactant is developed. An in vitro solution with surfactant based on this empirical data can be used to adjust the drug core in response to the surfactant of the tear film.

The drug cores may also be modified to utilize carrier vehicles such as nanoparticles or microparticles depending on the size of the molecule to be delivered such as latent-reactive nanofiber compositions for composites and nanotextured surfaces (Innovative Surface Technologies, LLC, St. Paul, Minn.), nanostructured porous silicon, known as BioSilicon.®., including micron sized particles, membranes, woven fivers or micromachined implant devices (pSividia, Limited, UK) and protein nanocage systems that target selective cells to deliver a drug (Chimeracore).

In many embodiments, the drug insert comprises of a thin-walled polyimide tube sheath with a drug core comprising latanoprost dispersed in Nusil 6385 (MAF 970), a medical grade solid silicone that serves as the matrix for drug delivery. The distal end of the drug insert is sealed with a cured film of solid Loctite 4305 medical grade adhesive. The drug insert may be placed within the bore of the punctum plug, the Loctite 4305 adhesive does not come into contact with either tissue or the tear film. The inner diameter of the drug insert can be 0.32 mm; and the length can be 0.95 mm. At least four latanoprost concentrations in the finished drug product can be employed: Drug cores can comprise 3.5, 7, 14 or 21 micrograms latanoprost, with per cent by weight concentrations of 5, 10, 20, or 30% respectively. Assuming an overall elution rate of approximately 100 ng/day, the drug core comprising 14 micrograms of latanoprost is configured to deliver drug for approximately at least 100 days, for example 120 days. The overall weight of the drug core, including latanoprost, can be about 70 micrograms. The weight of the drug insert including the polyimide sleeve can be approximately 100 micrograms.

In many embodiments, the drug core may elute with an initial elevated level of latanoprost followed by substantially constant elution of the latanoprost. In many instances, an amount of latanoprost released daily from the core may be below the therapeutic levels and still provide a benefit to the patient. An elevated level of eluted latanoprost can result in a residual amount of latanoprost or residual effect of the latanoprost that is combined with a sub-therapeutic amount of latanoprost to provide relief to the patient. In embodiments where therapeutic level is about 80 ng per day, the device may deliver about 100 ng per day for an initial delivery period. The extra 20 ng delivered per day can have a beneficial effect when latanoprost is released at levels below the therapeutic level, for example at 60 ng per day. As the amount of drug delivered can be precisely controlled, an initial elevated dose may not result in complications or adverse events to the patient.

In certain embodiments, the methods of the invention result in a percentage reduction in intraocular pressure of approximately 28%. In some embodiments, the methods of the invention results in a percentage reduction in intraocular pressure of approximately 27%, approximately 26%, approximately 25%, approximately 24%, approximately 23%, approximately 22%, approximately 21%, or approximately 20%. In certain embodiments, the methods of the invention result in a percentage reduction in intraocular pressure of at least 28%, at least 27%, at least 26%, at least 25%, at least 24%, at least 23%, at least 22%, at least 21%, or at least 20%.

In certain embodiments, the methods of the invention result in a reduction in intraocular pressure from baseline of about 6 mm Hg, about 5 mm Hg, about 4 mm Hg, about 3 mm Hg or about 2 mm Hg. In certain embodiments, the methods of the invention result in a reduction in intraocular pressure from baseline of at least 2 mm Hg, at least 3 mm Hg, at least 4 mm Hg, at least 5 mm Hg, or at least 6 mm Hg.

In an embodiment, the implants and methods of the invention provide a 90-day course of treatment. In some embodiments, effective levels of latanoprost release during the entire course of treatment. In a further embodiment, the variability in intraocular pressure over the course of treatment is less than about 1 mm Hg. In other embodiments, the variability in intraocular pressure over the course of treatment is less than about 2 mm Hg. In other embodiments, the variability in intraocular pressure over the course of treatment is less than about 3 mm Hg.

The implants described herein may be inserted into the superior punctum, the inferior punctum, or both, and may be inserted into one or both eyes of the subject.

### Eye Drop Adjunctive Compositions:

Eye drops are liquid drops used as a vector to administer therapeutic agents to the eye or to lubricate the eye or replace tears. The eye drop adjunctive compositions employed in the present invention are eye drops that administer therapeutic agents in addition to the described sustained release formulations.

Therapeutic agents administered as eye drop adjunctive compositions include any of the following or their equivalents, derivatives or analogs, including anti-glaucoma medications (e.g. ocular hypotensive drugs) including carbonic anhydrase inhibitors (CAIs, including but not limited to dorzolamide, brinzolamide and dorzolamide+timolol); Beta blockers including but not limited to levobunolol (Betagan), timolol (Betimol, Timoptic), carteolol (Ocupress), betaxolol (Betoptic) and metipranolol (OptiPranolol); Alpha-adrenergic agents including but not limited to apraclonidine (Iopidine) and brimonidine (Alphagan); Prostaglandin analogues including but not limited to: latanoprost (Xalatan), bimatoprost (Lumigan) and travoprost (Travatan); Miotics including but not limited to pilocarpine (Isopto Carpine, Pilocar); Epinephrine compounds; parasympathomimetics, hypotensive lipids, and combinations thereof; antimicrobial agents (e.g., antibiotic, antiviral, antiparacytic, antifungal, etc.); analgesics such as keterolac; corticosteroids or other anti-inflammatories (e.g., an NSAID such as diclofenac or naproxen); decongestants (e.g., vasoconstrictors); agents that prevent or modify an allergic response (e.g., antihistamines such as olopatadine, cytokine inhibitor, leucotriene inhibitor, IgE inhibitor, immunomodulator or immunosuppressants such as cyclosporin); mast cell stabilizers; cycloplegics or the like.

The eye drop adjunctive compositions employed in the present invention may contain, in addition to the therapeutic agents described above, one or more other components that are commonly present in ophthalmic solutions, for example, tonicity adjusting agents; isotonizing agents, buffers, pH regulators, preservatives and chelating agents. Isotonizing agents include sodium chloride, mannitol, sorbitol and glycerol; buffers include phosphates, boric acid, acetates and citrates; pH regulators include hydrochloric acid, acetic acid and sodium hydroxide; preservatives include p-oxybenzoates, benzalkonium chloride, chlorhexidine, benzyl alcohol, sorbic acid or salt thereof, thimerosal and chlorobutanol; chelating agents include sodium edetate, sodium citrate and condensed sodium phosphate. The eye drop adjunctive compositions may incorporate viscolyzer and/or suspending agents. Viscolyzer and/or suspending agents include methyl cellulose, carmellose or salts, hydroxyethyl cellulose, sodium alginate, carboxyvinyl polymer, polyvinyl alcohol and polyvinylpyrrolidone. Surfactants such as polyethylene glycol, propylene glycol, polyoxyethylene hydrogenated castor oil and polysorbate 80 may be incorporated in the eye drop adjunctive compositions.

The eye drop adjunctive compositions are formulated as eye-drops and sold in a wide range of small-volume containers from 1 ml to 30 ml in size. Such containers can be made from HDPE (high density polyethylene), LDPE (low density polyethylene), polypropylene, poly(ethylene terepthalate) and the like. Flexible bottles having conventional dispensing tops are especially suitable for use with the present invention. The eye drop adjunctive compositions of the invention are used by instilling, for example, about one (1) or two (2) or three (3) drops in the eye(s).

The pH of the eye drop adjunctive compositions may be maintained within the range of pH=5.0 to 8.0, preferably about pH=6.0 to 8.0, more preferably about pH=6.5 to 7.8, most preferably pH values of greater than or equal to 7; suitable buffers may be added, such as borate, citrate, bicarbonate, tris(hydroxymethyl)aminomethane (TRIS-Base) and various mixed phosphate buffers, and mixtures thereof.

The eye drop adjunctive compositions suitable for use in the present invention may also be useful as a component of a cleaning, disinfecting or conditioning solution and/or composition for contact lenses. Such solutions and/or compositions also may include, antimicrobial agents, surfactants, toxicity adjusting agents, buffers and the like that are known to be used components of conditioning and/or cleaning solutions for contact lenses.

The invention can be described by the following non-limiting examples.

### Example 1

Implant: The Punctum Plug Drug Delivery System (PPDS) may consist of a drug insert configured to be placed in a suitable commercially available punctum plug with a pre-existing bore. All materials used in the construction of the drug insert are medical grade materials that pass a battery of safety/toxicity tests. The drug insert is a thin-walled polyimide tube that is filled with latanoprost dispersed in Nusil 6385, a cured medical grade solid silicone. The cured silicone serves as the solid, non-erodible matrix from which latanoprost slowly elutes. The drug insert is sealed at the distal end with a cured film of solid Loctite 4305 medical grade adhesive (cyanoacrylate). The polyimide sleeve is inert and, together with the adhesive, provides structural support and a barrier to both lateral drug diffusion and drug diffusion through the distal end of the drug insert. The drug insert is seated in the bore of the punctum plug and is held in place via an interference fit. The assembled system is packaged and sterilized.

Eye drop adjunctive composition: Xalatan® latanoprost ophthalmic solution is a commercially available product indicated for the reduction of elevated IOP. The amount of latanoprost in the commercially available product Xalatan® is approximately 1.5 micrograms/drop. Xalatan® is supplied as a 2.5 mL solution in a 5 mL clear, low density polyethylene (PET) bottle with a clear low density PET dropper tip, a turquoise high density PET screw cap, and a tamper-evident clear low density PET overcap. Inactive ingredients of Xalatan® are benzalkonium chloride (preservative), sodium chloride, sodium dihydrogen phosphate monohydrate, disodium hydrogen phosphate anhydrous, and water.

Procedures: A punctum plug delivery system is inserted into one punctum of each eye of a patient having ocular hypertension. If intraocular pressure is not reduced significantly within four weeks of insertion, the eye drop adjunctive composition is administered once or twice daily for five days. Thus, the eye drop adjunctive composition can be administered anytime within the first four weeks of plug insertion, including concomitantly with plug insertion, a day to several days after insertion, or a week to four weeks after insertion, at the discretion of the practitioner. Thus, the eye drop adjunctive composition is administered at a dose of approximately 1.5 or 3.0 micrograms per day. In some instances, the delivery system is placed in the inferior punctum after an appropriate washout period, as defined in Table 2 below. If during subsequent visits the punctum plug system is not present a replacement device may be inserted.

Placement and removal of the Punctum Plug Drug Delivery System is accomplished in the same manner as for other commercially available punctum plugs. Generally, for placement the size of punctal plug to be used is determined by using suitable magnification or, if provided, using a sizing tool that accompanies the punctum plug. The patient's punctum is dilated if necessary to fit the punctum plug. A drop of lubricant is applied if necessary to facilitate placement of the plug into the punctum. Using an appropriate placement instrument the plug is inserted into the superior or inferior punctum of the eye. After placement, the cap of the plug is visible. This process is repeated for the patient's other eye. For removal of the implant, small surgical forceps are used to securely grasp the plug at the tube section below the cap. Using a gentle tugging motion the plug is gently retrieved.

TABLE 2
Recommended Washout Period

| Drug Class | Sample Agent(s) | Washout Period | |
|---|---|---|---|
| Prostaglandin analogs | Latanoprost (Xalatan), | 4 | weeks |
| | Bimatoprost (Lumigan), | | |
| | Travoprost (Travatan) | | |
| Beta blocker | Betaxolol (Betoptic) | 3 | weeks |
| | Timolol (Betimol) | | |
| Adrenergic agonists | Apraclonidine (Iopidine) | 2 | weeks |
| | Dipivefrin (Propine) | | |
| All other IOP lowering | Brinzolamide (Azopt) | 72 | hours |
| medications | Dorzolamide (Trusopt) | | |
| | Pilocarpine (Pilocar) | | |

During the 12-week course of treatment, intraocular pressure is measured by Goldmann applanation tonometry. Both a topical anesthetic and fluorescein are applied. This is accomplished by use of a combination product (e.g., Fluress®, benoxinate and fluorescein), or by separate application of a local anesthetic and fluorescein for corneal assessments. Immediately thereafter, intraocular pressure is measured using an applanation method.

### Example 2

The punctum plug delivery system implant and eye drop adjunctive composition are the same as in Example 1. The eye drop adjunctive composition is administered once or twice daily for two weeks prior to insertion of the punctum plug delivery system, with no washout period between the two week administration of the eye drop adjunctive composition and the insertion of the implant. The implant remains inserted in the punctum for up to twelve weeks. Intraocular pressure is monitored as in Example 1.

### Example 3

The punctum plug delivery system implant and eye drop adjunctive composition are the same as in Example 1. The eye drop adjunctive composition is administered once or twice daily for five days, beginning on the same day as the punctum plug delivery system is inserted. The punctum plug delivery system remains in the punctum for up to twelve weeks. Intraocular pressure is monitored as in Example 1.

### Example 4

Subjects are treated bilaterally in the lower puncta with a punctum plug delivery system (PPDS) containing 14 or 21 micrograms of latanoprost. The PPDS is replaced approximately every 12 weeks (3 months) for 3 cycles of treatment, resulting in a total duration of 9 months of treatment with the PPDS. If the intraocular pressure has increased to an uncontrolled level, the practitioner may replace the PPDS sooner. Removal of the PPDS (at the end of a cycle, for example) and insertion of a new pair of PPDS should occur on the same day. In the first cycle, subjects have follow-up visits every week for the first 4 weeks and biweekly thereafter until Week 12, with a visit window for each visit of ±3 days, relative to the day 0 visit of the treatment. In subsequent cycles, follow-up visits are scheduled for weeks 2, 6 and 12. Intraocular pressure is determined by Goldmann applanation tonometry measurements and is calculated as the average of values from both eyes, unless a PPDS has been lost. If intraocular pressure has not been controlled to 22 mmHg or less within the first 4 weeks of the first treatment cycle, then a 5-day adjunctive course of Xalatan® (0.005% latanoprost ophthalmic solution) eye drop adjunctive composition is initiated. Thus, the Xalatan® can be administered anytime within the first four weeks of plug insertion, including concomitantly with plug insertion, a day to several days after insertion, or a week to four weeks after insertion, at the discretion of the provider. The Xalatan® drops are administered once daily and as directed in the package insert. Subjects have a visit 1 week after initiating the Xalatan® therapy; therefore if a visit is not already scheduled for this time then the subject is brought in for an unscheduled visit to check IOP.

### BIBLIOGRAPHY

1. Anderson D R, Chauhan B, Johnson C, Katz J, Patella V M, Drance S M. Criteria for progression of glaucoma in clinical management and in outcome studies. Am J Ophthalmol 2000; 130(6):827-829.
2. Bailey I L, Lovie J E. New design principles for visual acuity letter charts. Am J Optom Physiol Opt November 1976; 53(11):740-745.
3. Balaram M, et al. Efficacy and tolerability outcomes after punctal occlusion with silicone plugs in dry eye syndrome. Am J Ophthalmol. 2001; 131(1):30-36.
4. Coleman A L. Glaucoma. Lancet 1999;354:1803-10.
5. Fiscella R G, Geller J L, Gryz L L, Wilensky J, Viana M: Cost considerations of medical therapy for glaucoma. Am J Ophthalmol 1999; 128:426.
6. Gordon M O, Kass M A. The Ocular Hypertension Treatment Study: design and baseline description of the participants. Arch Ophthalmol 1999; 117(5):573-583.
7. Javitt J C, Metrick S, Wang F: Costs of glaucoma in the United States. Invest Ophthalmol Vis Sci 1995; 36:S429.
8. Kim B M, et al. Pyogenic granulomas after silicone punctal plugs: a clinical and histopathologic study. Am J Ophthalmol. 2005; 139(4):678-684.
9. Kobelt-Nguyen G, Gerdtham U G, Alm A: Costs of treating primary open-angle glaucoma and ocular hypertension: a retrospective, observational two-year chart review of newly diagnosed patients in Sweden and the United States. J Glaucoma 1998; 7:95.
10. Lichter P R, Musch D C, Gillespie B W, et al. Interim Clinical Outcomes in The Collaborative Initial Glaucoma Treatment Study Comparing Initial Treatment Randomized to Medications or Surgery. Ophthalmology 2001;108:1943-53.
11. Musch D C, Lichter P R, Guire K E, Standardi C L, The CIGTS Study Group. The Collaborative Initial Glaucoma Treatment Study Design, Methods, and Baseline Characteristics of Enrolled Patients. Ophthalmology 1999; 106:653-662.
12. Norell S E, Granstrom P A. Self-medication with pilocarpine among outpatients in a glaucoma clinic. Br J Ophthalmol. February 1980;64(2):13-41.
13. Quigley H A: The number of persons with glaucoma worldwide. Br J Ophthalmol 1996; 80:389.
14. Regnier, et al. Ocular Effects of Topical 0.03% Latanoprost Solution in Normotensive Feline Eyes. Vet Ophthalmol 2006; 9(1):39-43.
15. Thylefors B, Negrel A-D, Pararajasegaram R, Dadzie K Y: Global data on blindness. Bull World Health Org 1995; 73:115-121.
16. Whitcup S M, et al. A randomized, doube masked, multicenter clinical trial comparing latanoprost and timolol for the treatment of glaucoma and acular hypertension. Br J Ophthalmol 2003; 87:57-62.
17. Winfield A J, et al. A study of the causes of non-compliance by patients prescribed eyedrops. Br J Ophthalmol. August 1990;74(8):477-80.
18. Woodward D F, et al. Pharmalogical Characterization of a Novel Antiglaucoma Agent, (AGN 192024). J Pharmacology and Experimental Therapeutics 2003; 305(2):772-785.
19. Xalatan® 0.005% (50 microgram/mL) prescribing information. Division of Pfizer Inc., New York, N.Y.: Pharmacia & Upjohn Company; 2007. http://www.xalatan.com/consumer/prescribininfo.asp. Accessed Oct. 1, 2007.

The above Detailed Description includes references to the accompanying drawings, which form a part of the Detailed Description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable Inconsistencies, the usage in this document controls.

The above description is intended to be illustrative, and not restrictive. For example, the above-described examples (or one or more features thereof) can be used in combination with each other. Other embodiments can be used, such as by one of ordinary skill in the art upon reviewing the above description. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

Concentrations, amounts, percentages, time periods, etc., of various components or use or effects of various components of this invention, including but not limited to the drug core, indications of reduction in IOP, and treatment time periods, are often presented in a range or baseline threshold format throughout this patent document. The description in range or baseline threshold format is merely for convenience and brevity and should not be construed as an inflexible limitation on the scope of the invention. Accordingly, the description of a range or baseline threshold should be considered to have specifically disclosed all the possible subranges as well as individual numerical values within that range or above that baseline threshold. For example, description of a drug core having a drug or other agent concentration range of 3.5 micrograms to 135 micrograms should be considered to have specifically disclosed subranges, such as 5 micrograms to 134 micrograms, 6 micrograms to 132 micrograms, 40 micrograms to 100 micrograms, 44 micrograms to 46 micrograms, etc., as well as individuals numbers within that range, such as 41 micrograms, 42 micrograms, 43 micrograms, 44 micrograms, 45 micrograms, 46 micrograms, 47 micrograms, 48 micrograms, etc. This construction applies regardless of the breadth of the range or baseline threshold and in all contexts throughout this disclosure.

What is claimed is:

1. A method to reduce intraocular pressure in an eye of a patient in need thereof, the method comprising administering to the eye of the patient a sustained release formulation comprising latanoprost and a pharmaceutically acceptable vehicle and administering an eye drop adjunctive composition, wherein the sustained release formulation releases latanoprost continuously for at least 90 days from a punctum plug delivery system.

2. The method of claim 1 wherein the eye drop adjunctive composition comprises an ocular hypotensive drug selected from the group consisting of carbonic anhydrase inhibitors, beta blockers, alpha-adrenergic agents, prostaglandin analogues, miotics and epinephrine compounds.

3. The method of claim 2 wherein the eye drop adjunctive composition comprises a prostaglandin analogue.

4. The method of claim 3 wherein the prostaglandin analogue comprises latanoprost.

5. The method of claim 1 wherein the eye drop adjunctive composition is administered once daily for less than about 10 days.

6. The method of claim 5 wherein the eye drop adjunctive composition is administered once daily for about 5 days.

7. The method of claim 1 wherein the eye drop adjunctive composition is administered once daily starting on the same day the punctum plug delivery system is inserted into a punctum of the patient.

8. The method of claim 1 wherein the eye drop adjunctive composition is administered once daily, starting within about four weeks after the punctum plug delivery system is inserted into a punctum of the patient.

9. The method of claim 1 wherein the eye drop adjunctive composition is administered once daily, starting about 90 days after the punctum plug delivery system is inserted into a punctum of the patient.

10. The method of claim 1 wherein the eye drop adjunctive composition is administered once daily, starting after removal of the punctum plug delivery system.

11. The method of claim 1 wherein the eye drop adjunctive composition is administered once daily, starting approximately five days before the punctum plug delivery system is inserted into a punctum of the patient.

12. The method of claim 1 wherein the eye drop adjunctive composition is administered after a first punctum plug delivery system is removed and before a second punctum plug delivery system is inserted into a punctum of the patient.

13. The method of claim 1 wherein the reduction in intraocular pressure is maintained for a continuous period of time selected from the group consisting of: up to about 7 days, up to about 14 days, up to about 21 days, up to about 28 days, up to about 52 days, up to about 88 days, and up to about 105 days.

14. The method of claim 1 wherein the reduction in intraocular pressure is maintained for a continuous period of time of at least about 90 days.

15. The method of claim 13 or 14 wherein the reduction in intraocular pressure is at least about 25%.

16. The method of claim 1 wherein the intraocular pressure is reduced by at least 10% by about 1 day after latanoprost and eye drop adjunctive composition administration is initiated.

17. The method of claim 1 wherein the pharmaceutically acceptable vehicle comprises a sustained release matrix.

18. The method of claim 17 wherein the sustained release matrix is a non-biodegradable polymer.

19. The method of claim 18 wherein the non-biodegradable polymer comprises silicone.

20. The method of claim 1 wherein the punctum plug delivery system is inserted into at least one punctum of the patient.

21. The method of claim 20 wherein the punctum plug delivery system is inserted into one punctum of each of both eyes of the patient.

22. The method of claim 1 wherein the intraocular pressure is associated with ocular hypertension.

23. The method of claim 1 wherein the intraocular pressure is associated with glaucoma.

24. The method of claim 23 wherein the glaucoma is selected from the group consisting of primary open angle glaucoma, angle closure glaucoma, normal tension glaucoma and secondary glaucoma.

25. A method to treat elevated intraocular pressure comprising inserting a punctum plug delivery system into at least one punctum of a patient in need thereof and administering an eye drop adjunctive composition to an eye of the patient in need thereof, wherein the punctum plug delivery system comprises a sustained release agent supply containing about 14 micrograms of latanoprost, wherein the punctum plug delivery system remains inserted for at least about 90 days, and wherein the eye drop adjunctive composition is administered for up to about 14 days.

26. The method of claim 25, wherein the eye drop adjunctive composition is administered for about ten days.

27. The method of claim 25, wherein the eye drop adjunctive composition is administered for about five days.

28. The method of claim 25, wherein the punctum plug delivery system includes a cavity configured to house the sustained release agent supply in the form of a drug core.

29. A method to treat elevated glaucoma-associated intraocular pressure in a subject in need thereof, the method comprising: inserting a punctum plug delivery system into at least one punctum of the subject and administering an eye drop adjunctive composition to an eye of the subject, wherein the punctum plug delivery system comprises a plug body and a latanoprost insert; wherein the eye drop adjunctive composition comprises latanoprost; wherein the punctum plug delivery system provides the sustained release of latanoprost to the subject; and wherein the release of latanoprost from the punctum plug delivery system and the administration of the eye drop adjunctive latanoprost composition together result in a reduction in the intraocular pressure of the associated eye of at least 6 mm Hg.

30. A method to treat glaucoma in a subject in need thereof, the method comprising: inserting a punctum plug delivery system into at least one punctum of the subject in a single insertion procedure and administering an eye drop adjunctive composition to the corresponding eye of the subject at least once; wherein the eye drop adjunctive composition comprises latanoprost; wherein the punctum plug delivery system comprises a plug body and a latanoprost insert; and wherein the punctum plug delivery system provides the sustained release of latanoprost to the subject for at least about 90 days.

31. The method of claim 4 wherein the amount of latanoprost in a single drop of eye drop adjunctive composition is approximately 1.5 micrograms.

32. A kit comprising a first container comprising the punctum plug delivery system of claim 1, a second container comprising the eye drop adjunctive composition of claim 1, and instructions for use.
